(19) United States
(12) Patent Application Publication
(10) Pub. No.: US 2010/0286106 A1
(43) Pub. Date: Nov. 11, 2010

(54) METHODS AND APPARATUS FOR TREATING THE PROSTATE

(76) Inventors: Yigal Gat, Rumat-Gan (IL); Menachem Goren, Petach-Tikva (IL)

Correspondence Address: MARTIN D. MOYNIHAN d/b/a PRTSI, INC., P.O. BOX 16446, ARLINGTON, VA, 22215, US

(21) Appl. No.: 12/668,696

(22) PCT Filed: Jul. 13, 2008
(86) PCT No.: PCT/IL08/00973

§ 371 Date: Jun. 24, 2010

Related U.S. Application Data
(60) Provisional application No. 61/064,511, filed on Mar. 10, 2008.

(30) Foreign Application Priority Data
Mar. 10, 2008 (US) 11826283

(51) Int. Cl. A61K 31/56; A61M 29/00; C07C 255/50; C07C 233/12; C07J 53/00; A61K 31/277; A61K 31/16; A61P 35/00; A61P 13/08

(52) U.S. Cl. 514/178; 604/96.01; 558/413; 564/218; 552/511; 514/522; 514/629

(57) ABSTRACT

Treating and/or preventing prostate problems and/or metastases, by selectively occluding various groin vessels and tools for selective occluding of various groin veins, including, for example, the deferential vein. Optionally providing anti-androgen treatment after such occluding.

### RELATED APPLICATIONS

This application claims the benefit under 119(e) of 61/064,511, filed Mar. 10, 2008 by inter alia, Yigal Gat and the benefit under 120 of 11/826,283, filed Jul. 13, 2007, by inter alia, Yigal Gat. This application is also related to international patent applications, Attorney Docket Nos. 43700, Title: METHODS AND APPARATUSES FOR VASCULAR AND PROSTATE TREATMENT and 43699, Title: DIAGNOSIS AND TREATMENT OF VARICOCELE AND PROSTATE DISORDERS, filed in the PCT on even date with the instant application and sharing at least inventor Yigal Gat, and which teach methods and apparatus which may be useful in conjunction with the below description. The disclosure of all of these applications is incorporated herein by reference.

### FIELD OF THE INVENTION

Some embodiments of the invention relate, inter alia, to treatments of the prostate. Some embodiments relate to treatment of veins linked to the prostate venous system.

### BACKGROUND OF THE INVENTION

A prostate in an adult male may develop disorders such as benign prostate hyperplasia (BPH) or prostate cancer.

FIG. 1 schematically illustrates a typical testicular and prostate venous drainage system of a human male. One drainage path from testes 104 comprises a pampiniform plexus 118 to a left internal spermatic vein 102 or right internal spermatic vein 130 that lead towards an inferior vena cava 106 through one-way valves 108. Normally, valves 108 facilitate venous blood flow upwards towards an inferior vena cava 106, and inhibit back flow down to a testis 104.

Another drainage path comprises a sequence of a pampiniform plexus 118 to a deferential vein 110, a vesicular vein 112, an internal iliac vein 114 and a common iliac vein 116 towards an inferior vena cava 106. The latter path is shared by a prostate 120 drainage path from a vesicular plexus 128 towards vesicular vein 112 and onwards.

Arteries 122 supply arterial blood to microcirculation vessels 124 of prostate 120 and microcirculation vessels 126 of testes 104.

FIG. 2 schematically illustrates typical testicular and prostate venous drainage paths in a normal left side of a human male where the arrows directions illustrate the venous blood flow as described above.

Since one-way valves 108 in internal spermatic vein 102 block back flow down to testes 104, they isolate hydrostatic pressure from the sections between them, so that a typical pressure at an entry 142 to left internal spermatic vein 102 is about 5-6 mmHg and may be somewhat lower at an entry 144 to right spermatic vein 130.

The venous blood emerging from the testes has, relative to other regions of the venous system, high concentration of testosterone secreted by the testes, and particularly free testosterone that eventually dilutes in the blood circulation and binds with proteins to form a bound serum testosterone.

The following articles relate in general to the subject of varicocele, male infertility and treatment and/or venous embolism.

Gat, Y., Zukerman, Z., Bachar, G. N., Feldberg, D., Gornish M. Adolescent varicocele: Is it a unilateral disease? Urology (2003), 62:742-746; Editorial Comment. 746 Reply by the Authors 746-747

Gat, Y., Bachar, G. N., Zukerman, Z. Garnish, M. Varicocele: a Bilateral Disease. Fertil. Steril. (2004); 81:424-429. Editorial Comment in Journal of Urology, 2004 172(2) 790-791.

Gat, Y., Bachar, G., Zukerman, Z., Belenky, A., Gornish, M. Physical Examination May Miss the Diagnosis of Bilateral Varicocele. J. Urol. (2004), 172:1414-7. Editorial Comment 1239-40. 2nd Editorial Commentary and Authors' Reply, in J. Urol. 2005; June; 173(6):2208-2209

Gat, Y., Gornish, M., Belenky, A., Bachar, G. N. Elevation of serum testosterone and free testosterone after embolization of the internal spermatic vein for the treatment of varicocele in infertile men. Hum. Reprod. (2004); 19:2303-6. Editorial Comment in J Urol. (2005); 173(6):2079

Gat Y, Bachar G N, Everaert K, Levinger U, Gornish M. Induction of spermatogenesis in azoospermic men after internal spermatic veins embolization for the treatment of varicocele. Hum. Reprod. (2005); 20:1013-1017. Editorial Comment in J. Urol. (2005), Nov. 174(5), 1942

Gat Y., Chakraborty, J., Zukerman, Z., Garnish, M. Varicocele, Hypoxia, and Male Infertility. Fluid mechanics analysis of the impaired testicular venous drainage system. Hum. Reprod. (2005); 20:2614-2619. Editorial Comment in J. Urol. (2006), Apr. 17(4), 1454.

Siegel Y, Gat Y, Bather G N, Garnish M. A Proposed Anatomic Typing of the Right Internal Spermatic Vein: Importance for Percutaneous Sclerotherapy of Varicocele. Cardiovasc Intervent Radial. 2006 March-April; 29(2):192-7.

Discussion and Author Reply in Cardiovasc Intervent Radiol. 2007 April; 30(2):348-349.

Gat Y., Garnish, M., Chakraborty, J., Navon U., Bachar G N., Ben Shlomo I. Right Varicocele and Hypoxia: Crucial factors in Male Infertility. Fluid mechanics analysis of the impaired testicular venous drainage system. Reprod Biomed Online. 2006 October; 13(4):510-5

Levinger U, Garnish M, Gat Y, Bachar G. N. Is Varicocele prevalence increasing with age? Andrologia. June, 2007, (3): 77-80.

Belenky A, Bartal G, Gat Y, Bachar G N. Uterine artery embolization: a pilot study in a rabbit model. Fertil Steril. 2005 February; 83(2):487-90.

Bachar G N, Belenky A, Greif F, Atar E, Gat Y, Itkin M, Verstanding A. Initial experience with ovarian vein embolization for the treatment of chronic pelvic pain syndrome. Isr Med Assoc J. 2004 February; 6(2):122.

Weiss D B, Gottschalk-Sabag S, Bar-On E, Zukerman Z, Gat Y, Bartoov B. [Seminiferous tubule cytological pattern in infertile, azoospermic men in diagnosis and therapy] Harefuah. 1997 May 1; 132(9):614-8, 680. Hebrew.

Gat Y., Garnish M., 2006, Technical investigation including imaging procedure for the detection of Varicocele. In: Schill, Comhaire, Hargreave (eds.). Text Book of Anthology for the Clinician. Springer Edition 2006, pp 447-453.

Gat Yigal, Varicocele: A bilateral disease. Thesis Defense for the PhD in The Medical Sciences, 19, Dec. 2006, Ghent University Hospital, Ghent, Belgium.

### SUMMARY OF THE INVENTION

A broad aspect of some embodiments of the invention relates to the recognition that excessive hydrostatic pressure (abnormal high pressure) in the testicular and prostatic veins (e.g., close to or higher than testicular arterial pressure), due, for example, to impaired valves in the internal spermatic veins, can play a causative role in prostate disorders such as BPH, cancer, and/or testosterone deficiency, possibly as outlined below.

In some embodiments of the invention, occlusion of one or more veins in the abdomen or inguinal region reduces the excessive hydrostatic pressure and/or testicular venous backflow into the pampiniform plexus and/or the prostate.

In embodiments of the invention, the term ‘cancer’ or metastases thereof relate to abnormal cells that (a) develop and proliferate responsive to androgen, and/or (b) that are diminished or annihilated or suppressed, and in some cases healed, responsive to deficiency of androgen and/or responsive to treatment of androgen antagonist (antiandrogen). The androgen typically comprises testosterone or derivatives thereof, and in some cases particularly free testosterone or dehydrotestosterone (DHT).

An aspect of some embodiments of the invention relates to treatment procedures related to the prostate. In some embodiments of the invention, a treatment is directed to prostate cancer. In some embodiments of the invention, a treatment is directed to metastases of a prostate cancer. In some embodiments of the invention, a treatment is directed to forestalling and/or preventing the development of prostate disorders.

An aspect of some embodiments of the invention relates to combining pharmaceutical treatment of prostate cancer with venous blockage. In an exemplary embodiment of the invention, the use of venous blockage enhances an anti-androgen or other anti-cancer treatment. Optionally or alternatively, androgen levels may be reduced less than in the art, if a direct connection between testis and prostate is blocked. Optionally, it is a target of treatment to maintain a minimal serum level of androgens that is higher than in art, and, for example, less likely to cause bodily harm. In exemplary embodiments of the invention, new regimens and/or new dosage levels of existing anti-androgen drugs are provided. Optionally, anti-androgen treatments are avoided if the presence of venous backflow from a testis to a prostate is believed to exist. Optionally or alternatively, some embodiments of the invention allow one to avoid placing the prostate in a state of elevated but not hyper-elevated testosterone level, which state might enhance the production of metastases and/or androgen-resistant tumors. In an exemplary embodiment of the invention, the timing of the procedure(s) and application of anti-androgen or other chemotherapeutic treatment is selected so that the combined effect is synergetic and/or at least not interfering between the two types of treatment. In one example, radiation treatment is started when androgen levels in prostate are still high (and tissue possibly undergoing active proliferation) and after a time, for example, a few days or weeks, venous reflux is stopped and/or anti-androgen provided.

An aspect of some embodiments of the invention relates to apparatus for vein sclerotherapy and a method of operating thereof. In some embodiments of the invention, the apparatus comprises an intravascular catheter for sclerotherapy, designed to apply the sclerosing material into the opening of a branching blood vein. In an exemplary embodiment of the invention, the catheter extends an injection means sides into such a branch. Optionally or alternatively, the catheter is configured to detect the position of the branch optionally mechanically and/or using imaging.

An aspect of some embodiments of the invention relates to a tool designed for selective engagement of a vein, from an outside thereof. In some embodiments of the invention, the apparatus is configured to separate and occlude a vein in a subcutaneous operation, from outside the vein. Optionally, the apparatus comprises an extension which curves when it engages a vein.

In the specifications and claims, unless otherwise specified, the term ‘rich in’ denotes a concentration of a substance relative to and beyond a normal limit, such as twice or more than the normal limit.

In the specifications and claims, unless otherwise specified, the term ‘excessive’ or ‘high’ denotes a measure (e.g. a quantity or amount) relative to and beyond a normal limit, such as twice or more than the normal limit. For example, an excessive or a high pressure or a high concentration.

In the specifications and claims, unless otherwise specified, the term ‘normal’ denotes a measure (e.g. a quantity or amount) in a range as typically found in healthy persons or organs.

In the specifications and claims, unless otherwise specified, the term ‘distal’ denotes a direction towards the body internals and the term ‘proximal’ denotes the opposite direction with respect to a treatment apparatus.

As used herein, the term “treating”, when used in general terms includes abrogating, substantially inhibiting, slowing or reversing the progression of a condition, substantially ameliorating clinical or aesthetical symptoms of a condition or substantially preventing the appearance of clinical or aesthetical symptoms of a condition.

The terms above apply also to their inflections.

There is thus provided in accordance with an exemplary embodiment of the invention, a method for treating the prostate, comprising:

(a) positing the existence of abnormal cells that respond to an androgen in the prostate; and

(b) occluding an internal spermatic vein.

In an exemplary embodiment of the invention, the abnormal cells are localized in the prostate.

In an exemplary embodiment of the invention, the androgen comprises testosterone. Optionally, the testosterone comprises one or both of free testosterone and dehydrotestosterone.

In an exemplary embodiment of the invention, occluding an internal spermatic vein comprises occlusion of the left and right internal spermatic veins.

In an exemplary embodiment of the invention, the method comprises administration of antiandrogen medication. Optionally, the antiandrogen medication is administered locally to the prostate. Optionally or alternatively, the administration of antiandrogen follows the occlusion.

In an exemplary embodiment of the invention, occlusion of an internal spermatic vein comprises occlusion a junction between the internal spermatic vein and the pampiniform plexus.

In an exemplary embodiment of the invention, the method comprises application of at least one of chemotherapy, radiotherapy, brachytherapy, thermotherapy or cryotherapy.

There is also provided in accordance with an exemplary embodiment of the invention, a method for treating metastases, comprising:

(a) detecting prostate cancer metastases; and

(b) reducing venous pressure in the prostate while blocking reflux from testicular veins subject to abnormally high pressure.

In an exemplary embodiment of the invention, reducing pressure in the prostate comprises occluding a vein between the prostate and a pampiniform plexus. Optionally the vein is a least one of a deferential vein or a vesicular plexus. Optionally or alternatively, an abnormally high pressure is maintained in the internal spermatic veins.

In an exemplary embodiment of the invention, the method comprises comprising administration of antiandrogen medication. Optionally, the administration of antiandrogen follows the occlusion.

In an exemplary embodiment of the invention, the method comprises comprising application of at least one of chemotherapy, radiotherapy, brachytherapy, thermotherapy or cryotherapy.

There is also provided in accordance with an exemplary embodiment of the invention, a method for a preventive treatment of the prostate, comprising:

(a) assessing a risk for development of prostate disorder; and

(b) responsive to the risk, preventing venous reflux to the prostate.

In an exemplary embodiment of the invention, preventing venous reflux to the prostate comprises occluding an internal spermatic vein. Optionally, occluding an internal spermatic vein comprises occlusion of the left and right internal spermatic veins. Optionally or alternatively, prostate disorder comprises one of BPH and cancer.

In an exemplary embodiment of the invention, assessing comprises assessing varicocele. Optionally, varicocele is assessed by testicular temperature.

In an exemplary embodiment of the invention, assessing comprises determining at least one of PSA level, prostate size or venous anatomy.

There is also provided in accordance with an exemplary embodiment of the invention, an intravascular catheter configured to apply material sideways from a first vein into a second vein branching from the first vein, comprising:

(a) a tube having a lumen; and

(b) an orifice connecting the lumen and a longitudinal external side of the tube.

In an exemplary embodiment of the invention, the connection is substantially perpendicular to the longitudinal external side of the tube. Optionally or alternatively, the lumen is sealed at the distal end. Optionally or alternatively, the orifice is sufficiently close to the sealed end to enable material in the lumen to deflect from the lumen via the orifice outside the lumen.

In an exemplary embodiment of the invention, the catheter comprises at least one guiding element configured to support a positioning of the orifice in front of an opening of the second vein. Optionally, the at least one guiding element comprises a guide wire in the lumen movable into the orifice and the opening of the second vein. Optionally or alternatively, the at least one guiding element comprises at least one radio opaque element located about the perimeter of the orifice.

In an exemplary embodiment of the invention, the catheter comprises at least one expandable and retractable element capable of blocking material drainage along the outside of the catheter. Optionally, the expanded at least one element is configured to fixate the catheter to the first vein walls.

In an exemplary embodiment of the invention, the material comprises a sclerosant. Optionally or alternatively, the material comprises a coil.

There is also provided, in accordance with an exemplary embodiment of the invention, an apparatus configured for a combined separation and sealing of a vein, comprising a tube with a lumen, and configured to selectively curve and loop at a distal section thereof, said loop having an inner diameter of less than 2 mm, said tube having a width smaller than 2 mm. Optionally, the separation comprises separation of at least a part of the vein from connecting tissues and said distal section is configured for such separation. Optionally, the vein is a deferential vein and the connecting tissues comprise a vas deferens. Optionally or alternatively, said lumen is adapted for providing temperature induced sclerosis. In an exemplary embodiment of the invention, the apparatus comprises a filament in a distal section of the lumen, configured for heating the distal section of the tube.

In an exemplary embodiment of the invention, the apparatus comprises a control wire adapted to maneuver and shape the tube to enable separation of and looping around at least a part of the vein.

Optionally, the distal end is tapered to support a separation of at least a part of the vein from connecting tissues.

There is also provided in accordance with an exemplary embodiment of the invention, an apparatus configured for a combined separation and sclerosing of a vein, comprising:

(a) a tube having a lumen;

(b) a pair of arms shaped for gripping a vein mounted at the distal end of the tube; and

(c) a sclerosant injector adapted for insertion in the lumen and piercing the vein when said arms grip a vein.

In an exemplary embodiment of the invention, the arms are adapted to controllably open and close a grip. Optionally or alternatively, at least one arm is configured for separating at least a part of the vein from connecting tissues. Optionally or alternatively, the vein is a deferential vein and the connecting tissues comprise a vas deferens.

In an exemplary embodiment of the invention, the vein is the deferential vein.

In an exemplary embodiment of the invention, the apparatus comprises a control wire which controls said gripping.

There is also provided in accordance with an exemplary embodiment of the invention, a method for vein sclerosis, comprising:

(a) maneuvering a catheter to a first vein from which a second vein branches; and

(b) applying a material from an orifice at the side of the catheter into an opening of the second vein.

In an exemplary embodiment of the invention, maneuvering comprises positioning the orifice in front of an opening of the second vein. Optionally or alternatively, the method comprises fixing at least a part of the catheter to the first vein wall.

In an exemplary embodiment of the invention, the first vein is a vesicular vein. Optionally or alternatively, the first vein is a pampiniform plexus.

In an exemplary embodiment of the invention, the second vein is a deferential vein. Optionally or alternatively, the second vein is a vesicular plexus vein.

There is provided in accordance with an exemplary embodiment of the invention, a method for vein sealing, comprising:

(a) subcutaneously accessing a vein;

(b) separating at least a part of the vein from connecting tissues; and

(c) sealing the at least part of the vein.

In an exemplary embodiment of the invention, subcutaneously accessing a vein comprises a accessing via a tube. Optionally or alternatively, the vein is a deferential vein and the connecting tissues comprise a vas deferens. Optionally or alternatively, separating at least a part of the vein comprises curving an element around the vein and moving the element. Optionally or alternatively, separating at least a part of the vein comprises gripping the vein. Optionally or alternatively, sealing the vein comprises one of (i) heating the vein, (ii) cooling the vein, or (iii) introducing a sclerosant agent into the vein.

There is provided in accordance with an exemplary embodiment of the invention, and the use of an anti-androgen composition for treatment of prostate cancer in patients with a blocked differential vein.

There is provided in accordance with an exemplary embodiment of the invention, the use of a sclerosant for treatment of prostate cancer or BPH.

There is provided in accordance with an exemplary embodiment of the invention, the use of an anti-androgen composition for treatment of prostate cancer by achieving a prostatic androgen level of less than 20% of a normal level.

There is provided in accordance with an exemplary embodiment of the invention, the use of an anti-androgen composition for treatment of prostate cancer by achieving a serum androgen level of more than 10% of a normal level and less than 90% of a normal level.

There is provided in accordance with an exemplary embodiment of the invention, a method of calculating a dosage of an anti-androgen treatment for prostate cancer, comprising:

(a) determining a level of venous reflux from testis to prostate; and

(b) calculating a dosage of an anti-androgen treatment based on said determined level.

There is provided in accordance with an exemplary embodiment of the invention, the use of an anti-androgen composition for treatment of prostate cancer in patients with a blocked internal spermatic vein.

### BRIEF DESCRIPTION OF THE DRAWINGS

Non-limiting examples of embodiments of the present invention are described with reference to figures listed below. In the drawings which follow, identical and/or equivalent and/or similar structures, elements, or parts that appear in more than one drawing are generally labeled with the same numeral in the drawings in which they appear. Dimensions of components and features shown in the figures are chosen for convenience and clarity of presentation and are not necessarily shown to scale.

FIG. 1 schematically illustrates a typical testicular and prostate venous drainage system of a human male;

FIG. 2 schematically illustrates typical testicular and prostate venous drainage paths in a normal left side of a human male;

FIG. 3 schematically illustrates typical testicular and prostate venous drainage paths in a left side of a human male when the one-way valves in the internal spermatic vein do not function;

FIG. 4A schematically illustrates a catheter designed to align with the deferential vein (or another branching vein) for injecting an agent into the vein while preventing the agent from reaching other regions by an expandable element (shown in collapsed state), in accordance with seine embodiments of the invention;

FIG. 4B schematically illustrates a catheter designed to align with the deferential vein (or another branching vein) for injecting an agent into the vein while preventing the agent from reaching other regions by an expandable element (shown in expanded state), in accordance with some embodiments of the invention;

FIG. 4C schematically illustrates an orifice at a distal end of the catheter of FIGS. 4A and 4B, comprising radio-opaque elements around the orifice, enabling to position the orifice at an opening of a branching vein, in accordance with some embodiments of the invention;

FIG. 5A schematically illustrates an apparatus for deploying a coil (shown in a stretched state) in the deferential vein (or another branching vein), in accordance with some embodiments of the invention;

FIG. 5B schematically illustrates an apparatus for deploying a coil (shown in a coiled state) in the deferential vein (or another branching vein) after the coil is deployed, in accordance with some embodiments of the invention;

FIG. 6A schematically illustrates a section of the deferential vein and a section of the vas deferens as they are attached to each other;

FIG. 6B schematically illustrates a spike inserted between the deferential vein and the vas deferens, in accordance with some embodiments of the invention;

FIG. 6C schematically illustrates a spike separating the deferential vein from the vas deferens, in accordance with some embodiments of the invention;

FIG. 6D schematically illustrates a spike curling into a loop around the deferential vein separated from the vas deferens, in accordance with some embodiments of the invention;

FIG. 6E schematically illustrates a spike having a lumen with a control wire and filament with conductors, in accordance with some embodiments of the invention;

FIG. 7A schematically illustrates a tube with a hinged pair grippers at one end (shown in open position), in accordance with some embodiments of the invention;

FIG. 7B schematically illustrates a tube with a hinged pair grippers (shown in closed position around a vein) at one end, in accordance with some embodiments of the invention; and

FIG. 7C schematically illustrates a tube with hinged pair grippers (shown in closed position around a vein) at one end, and an injector inserted in the lumen piercing the vein, in accordance with some embodiments of the invention.

### DETAILED DESCRIPTION OF EMBODIMENTS OF THE INVENTION

### Overview

The left and right side of the testicular and prostate venous drainage system of a human male are similar, where notably the left internal spermatic vein flows into the left renal vein and the right spermatic vein flows into the inferior vena cava.

Unless otherwise specified, the descriptions and discussions and examples that follow apply to both the left and right side anatomies of a male.

Internal spermatic veins with incompetent or destroyed one way valves cannot maintain upstream venous flow, resulting in a blood column of about 35-40 cm in a normal adult male. The column exerts, relative to normal conditions, an excessive hydrostatic pressure at the lower parts of the internal spermatic veins and connecting vessels. The excessive hydrostatic pressure prevents venous blood in the pampiniform plexus (which drains the testis) from flowing upwards in the internal spermatic vein to the inferior vena cava on the right side and towards the renal vein on the left side. Rather, it has been realized, that under the excessive hydrostatic pressure, venous blood rich with testosterone from the testis is diverted to the deferential vein in a backward direction (retrograde, reflux), flows via the vesicular vein into the vesicular plexus, into the prostate venous plexus (‘Santorini’ plexus) and ultimately into and around the prostate. Possibly, other retrograde pressure and/or flow pathways exits, possibly depending on the personal anatomy.

The altered venous flow from the testis towards the prostate under the excessive hydrostatic pressure restricts the drainage of prostate veins, possibly leading to swelling (dilation) and/or hypertrophy of the prostate.

Furthermore, under normal physiologic condition, free testosterone (secreted by the testes) drains to the general blood circulation where it is diluted and binds (about 98%) to proteins such as SHBG (serum hormone binding globulin) and albumin. With abnormal (or damaged) internal spermatic vein valves, however, the reverse flow diverts free testosterone from its production site in the testis directly to the prostate, by direct flow and/or by diffusion along the veins, greatly increasing the concentration of testosterone in the gland, and particularly free testosterone or possibly other compounds of testosterone or other substances, to an excessive level far above normal levels (typically beyond the normal level of about 17 nmol/l total and 10 nmol/l of free testosterone). It is theorized that the excessive level of free testosterone in the prostate stimulates cell proliferation (such as BPH) and/or cancer. For example, it known that the growth of prostate cancer cells typically initially requires male hormones, such as testosterone, as in ‘Fact Sheet Prostate Cancer, National Institute of Health, September 2007’, incorporated herein by reference.

In some embodiments of the invention, a vessel between the prostate and the pampiniform plexus is occluded, for example, for forestalling and/or therapy of BPH, prostate cancer and/or metastases by preventing or impeding the reflux of testicular venous blood to the prostate. Optionally, the vessel comprises the deferential vein or the vesicular plexus.

In some embodiments of the invention, the occlusion of the deferential vein or the vesicular plexus disconnects the prostate venous system from the excessive pressure at the pampiniform plexus and/or internal spermatic vein, such that:

(a) the testicular venous blood rich in testosterone is impeded from reaching the prostate, thus maintaining a substantially normal blood pressures and testosterone concentration in the prostate, reducing the probability of developing disorders in a substantially healthy prostate; and

(b) prostate venous blood can drain via the vesicular plexus (or other veins) up towards the vena cava, relieving the prostate of the excessive pressure and high testosterone concentration that might have developed due to the reflux, allowing an ailing prostate to recover, at least partially.

In some embodiments of the invention, occluding only the deferential vein (DV) or the vesicular plexus (VP) rather than, additionally or separately, occluding the internal spermatic vein with the incompetent one-way valves may have advantages for one or more of the following reasons:

(a) elimination of the introduction of a catheter or wire in the internal spermatic vein against the counter direction of the one-way valves in the internal spermatic vein;

(b) the deferential vein or the vesicular plexus can be reached via the femoral vein and iliac veins to the vesicular vein, which may be easier than reaching up the abdomen veins and down the internal spermatic vein;

(c) the deferential vein or the vesicular plexus can be accessed percutaneously; and

(d) the testicular venous system is substantially not affected (optionally treated separately or in a later time, optionally according to assessment of the patient health), possibly preventing an increase in androgens to potential metastases.

In some embodiments of the invention, occlusion of the deferential vein or the vesicular plexus is indicated in case of prostate cancer and/or metastases, for example, to let the prostate recover as described herein. Optionally, occlusion of the deferential vein or the vesicular plexus is accompanied with androgen medication and/or chemotherapy and/or radiotherapy and/or thermotherapy, optionally to reduce metastasis.

In some embodiments, the varicocele is treated, which prevents excessive pressure on the testies drainage as well, allowing drainage through other paths.

The deferential vein is a thin-walled narrow and delicate vein normally about 0.1 width of about 0.3-0.5 mm. The vesicular plexus branches are about the same widths and respond similarly to the excessive pressure.

Additionally, the deferential vein typically extends alongside the vas deferens, also a thin-walled narrow and delicate conduit.

In some embodiments of the invention, an opening of the deferential vein or the vesicular plexus is occluded. In such a case, optionally, care is exercised to avoid occlusion of other veins, such as the cremasteric vein.

In some embodiments of the invention, treating the thin deferential vein or the vesicular plexus may require care to avoid damage to the vein or other veins or the vas deferens, and may require special equipment for occlusion. In some cases, for example, older and/or sterile patients, damage to the vas deferens is not considered critical. Alternatively, an intentional combined act of sterilization and blocking the deferential vein is carried out.

An aspect of some embodiments of the invention relates to a procedure for treating prostate cancer, optionally and particularly localized at the prostate, comprising occlusion of an internal spermatic vein and optionally administration of antiandrogen medication.

An aspect of some embodiments of the invention relates to a procedure for treating metastases of prostate cancer, comprising occlusion of a deferential vein or the vesicular plexus, and optionally administration of antiandrogen and/or other medication.

An aspect of some embodiments of the invention relates to a procedure for forestalling and/or preventing prostate disorders (e.g. BPH or cancer) and/or testicular disorders (e.g. reduced testosterone production and delivery), comprising occlusion of a vein such as the internal spermatic vein and/or deferential vein and/or the vesicular plexus.

An aspect of some embodiments of the invention relates to a procedure for forestalling and/or treating disorders of the prostate (e.g. BPH or cancer), optionally and particularly avoiding recurrence of the disorder by reducing hydrostatic pressure in venous bypasses that may develop, comprising occlusion the junction of the internal spermatic vein and the pampiniform plexus.

An aspect of some embodiments of the invention relates to apparatus for sclerosing of the deferential vein or the vesicular plexus without inflicting damage to other vessels such as the cremasteric vein or vas deferens. Optionally, the apparatus is configured to treat other veins branching from another vein.

In some embodiments of the invention, the apparatus supports reaching the vesicular vein and/or the deferential vein from the internal iliac vein.

In some embodiments of the invention, the apparatus comprises an intravascular catheter for sclerotherapy, configured to apply the sclerosing agent sideways into an opening of the deferential vein or the vesicular plexus, optionally limiting or avoiding agent flow to another region.

An aspect of some embodiments of the invention relates to a medical tool configured to perform both separation and occlusion of a vein.

In some embodiments of the invention, the tool forms a loop or partial loop around a vein such as the deferential vein or a branch of the vesicular plexus, separating a part of the vein from other tissues or organs such as the vas deferens. In some embodiments of the invention, the tube comprises heating elements for inducing occlusion of the vessel. Optionally or additionally, the tube is flushed with hot or cold fluid at a temperature sufficient for causing the vessel's occlusion. In some embodiments of the invention, the vein is treated by the wire subcutaneously in a laparoscopic-like method.

In some embodiments of the invention, the apparatus comprises grippers configured to hold a vein such as the deferential vein or a branch of the vesicular plexus, separating a part of the vein from other tissues or organs such as the vas deferens. Optionally, the grippers are mounted on a tube having a lumen for inserting an injector for applying a sclerosant into the gripped vein. In some embodiments of the invention, the vein is treated by the gripper subcutaneously in a method similar to laparoscopy.

The section headings used herein are intended for convenience only and not intended to be necessarily limiting.

### Anatomy and Labeling

In the discussion below, unless otherwise specified, referring to anatomy elements applies to the left and right sides.

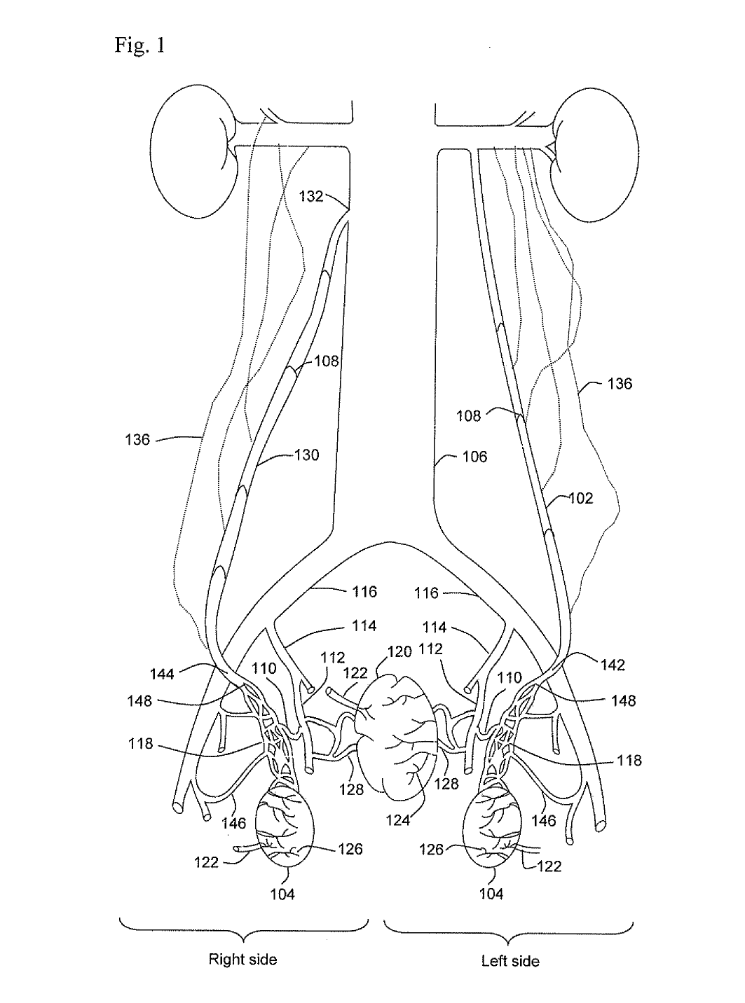

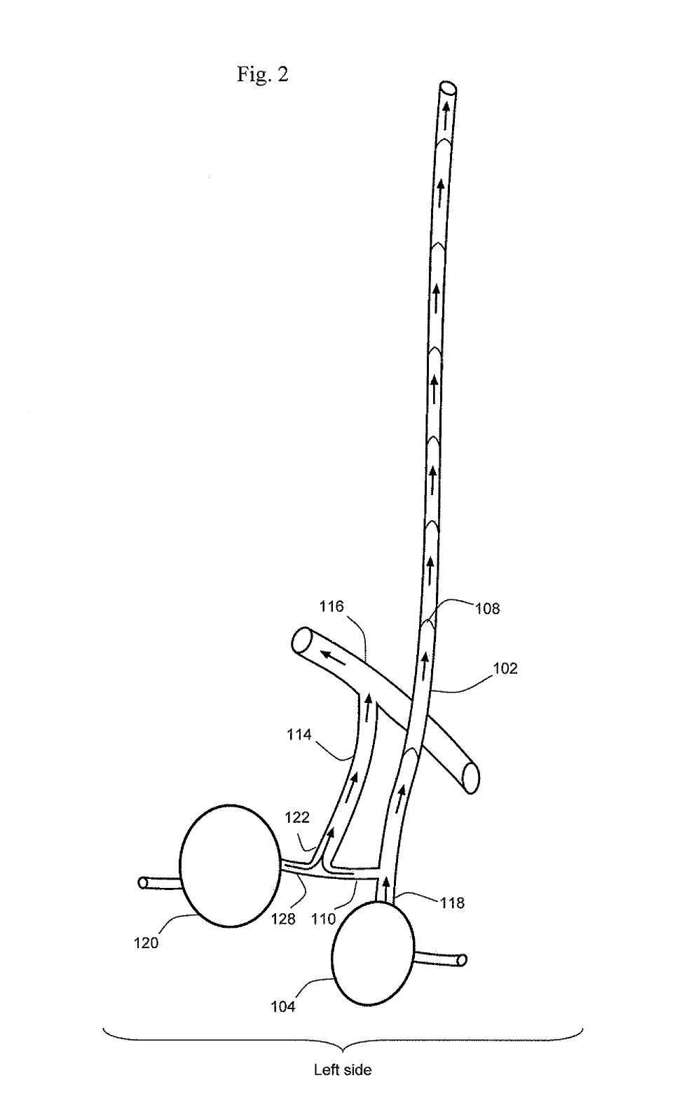

The labeling of the anatomy elements, unless otherwise specified, is according to FIG. 1 to FIG. 3.

In some discussions, anatomy elements are illustrated in other figures and may have labels with respect to the corresponding figure.

### Some Effects of High Hydrostatic Pressure

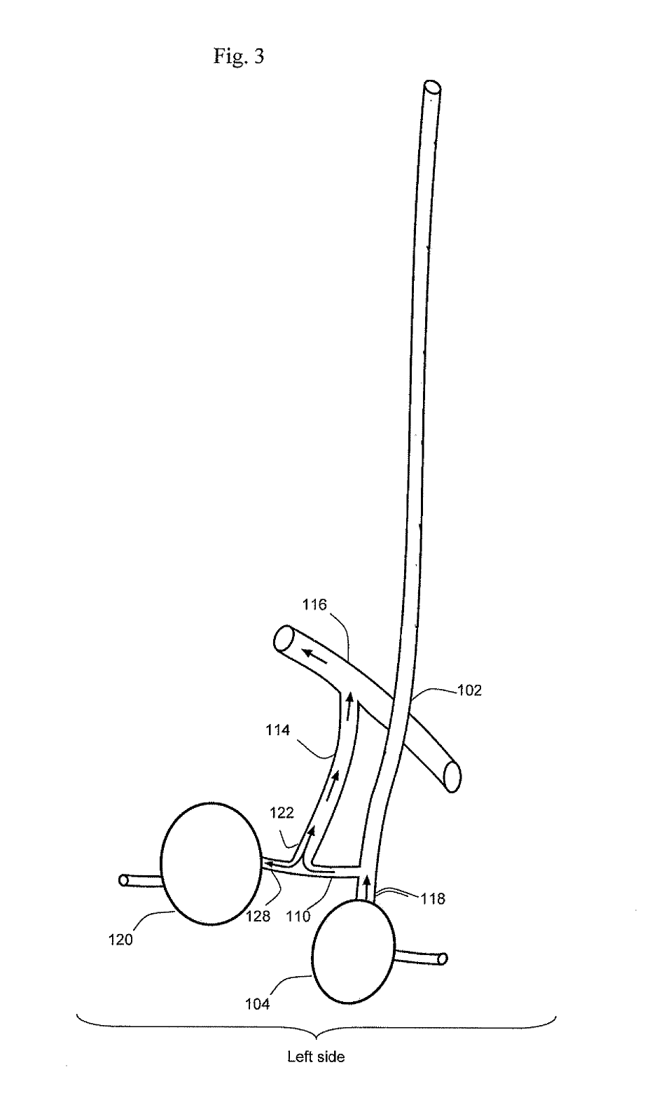

FIG. 3 schematically illustrates typical testicular and prostate venous drainage paths in a left side of a human male when the one-way valves in the internal spermatic vein do not function normally, for example, due to mechanical deterioration such as weakening of valve substance, wearing away or aging effects. The following is intended to be a non-limiting description of processes which may happen with regard to venous flow. The methods and apparatus of some embodiments of the invention may be used to independently of the correctness and/or completeness of the following discussions. Some embodiments are used and/or modified according to the flow paths and/or pressures described herein.

Since one-way valves 108 in internal spermatic vein 102 or 130 do not block back flow (retrograde flow, reflux) down to testes 104, internal spermatic veins 102 or 130 form continuous columns of blood in which hydrostatic pressure develops up to approximately 31 mmHg at entry 142 to left internal spermatic vein 102 approximately 27 mmHg at entry 144 to right internal spermatic vein 130 (typically about 4-6 fold the typical pressure in ordinary conditions) when the patient is in an upright position such as standing. This excessive hydrostatic pressure, or a pressure of similar magnitude, may exist in vessels connecting to internal spermatic vein 102, such as deferential vein 110 or pampiniform plexus 118, since, according to Bernoulli's law of connecting vessels, the pressure propagates from the testicular to the prostate venous drainage systems and hydro-dynamically equilibrates between both drainage systems. The pressure may diminish as vessels are further away from entry 142 or 144, but may be still more than the normal range of about 5 mmHg. The excessive pressure at entry 142 or 144 and nearby vessels will be denoted as ‘EP’.

The excessive high pressure EP inhibits the drainage of the venous blood from testes 104 and pampiniform plexus 118 up internal spermatic vein 102. Rather, the pressure differentials urge the testicular venous blood, rich in free testosterone (about 130 fold above serum level), towards vesicular plexus 128 and onwards to prostate 120; the increased pressure may also result in high pressure in the prostate and/or constrained drainage of venous blood from the prostate.

As the blood still circulates, and flow via internal spermatic vein 102 is obstructed, venous blood from the testis is drained, at least partly, via other paths, such as deferential vein 110, a scrotal vein 146 or by-pass veins 136 that might have developed, possibly due to the excessive pressure.

As the prostate is congested with blood at high pressure, pressure equilibrium is reached between the prostate and the incoming backflow, and a constrained circulation is maintained as venous blood drains via vesicular plexus 128 and vesicular vein 112, and to some extent, via other veins. In some cases, prostatic veins may develop due to the high pressure in the prostate.

The excessive pressure EP may produce at least some of the following effects on the prostate:

(a) The venous blood that is diverted towards prostate 120 and congests and enlarges (swells) prostate 120. The swelling of prostate 120 may be manifested, at least partially, as BPH or other prostate problems.

(b) The venous blood from the testes 106, rich in free testosterone (up to 5 to 10 fold, or higher such as 50 to 100 fold) bathes the prostate gland cells with free testosterone, effecting benign prostate hyperplasia (BPH). About 90% of the free testosterone is irreversibly converted to dehydrotestosterone (DHT), which has about five fold higher affinity for androgen receptors than free testosterone and may effect an accelerated proliferation of prostate cells. It should be noted that due to the short passage from testes 104 to prostate 120 (about 10-15 cm), possibly only a small amount of free testosterone is bound to SHBG or albumin before bathing the prostate receptors.

Furthermore, the molecule of free testosterone (and DTH) is smaller than a bound testosterone molecule (or ligand), and may more easily diffuse through the prostate gland interstitium to reach the glandular tissue cells, while the bound testosterone is blocked.

Measurements in patients with varicocele have shown an amount of testosterone that is 100 fold normal values and free testosterone that is 133 fold normal values at the junction of the ISV and DV, which backflow towards the prostate.

(c) The excessive pressure EP and congestion of the prostate inhibits or reduces arterial blood from entering microcirculation 124 of the prostate and disrupts its biological balance, possibly inducing a hypoxic state. The excessive amounts of testosterone and DHT present in the prostate and/or hypoxic state may induce an accelerated proliferation of prostate cells and/or promote the development of cancer. It is hypothesized that the extreme concentration of free testosterone (up to or more than 100 fold relative to normal) in the prostate may overload the DNA hormonal feed back system, and increase the probability of mutations in the accelerated cells divisions.

The excessive pressure EP in the ISV may produce at least some of the following effects on the testes:

(a) The excessive venous pressure EP inhibits or reduces normal arterial blood flow from entering microcirculation 126 of the testes. The blood stagnates to at least some extent, and oxygenated arteriolar blood cannot flow normally into the testis, resulting in degenerative processes in the testes tissues which diminish its testosterone production and/or cause infertility.

(b) The impaired testosterone production, resulting in reduced testosterone in the blood serum, may effect aging expressions or symptoms.

### Remedy of Some Pressure Induced Effects

In exemplary embodiments of the invention, one or more of the adverse states and effects described above may be avoided, delayed, alleviated and/or repaired, at least to some degree, by reducing or eliminating the excessive pressure EP. Reducing the excessive pressure reduces or eliminates the back flow (reflux) of venous blood, rich in free testosterone, from the testes to the prostate and/or is used to reduce EP on the testis themselves.

In exemplary embodiments of the invention, the reflux that has effected the excessive hydrostatic pressures EP is prevented or impeded by occlusion (e.g. by embolization and/or sclerosis) of left internal spermatic vein 102 and/or right internal spermatic vein 130. Optionally and additionally, some or all veins through which the reflux flows, such as deferential vein 110 and vesicular plexus 128, are occluded. Optionally or additionally, bypass veins 136 that might have developed connecting parts of internal spermatic vein 102 and/or 130, or connecting internal spermatic vein 102 and/or 130 to the renal vein are occluded, too.

Once the excessive pressure EP is reduced or eliminated by occluding internal spermatic veins 102 and/or 130, venous blood from the testes may use alternative paths to drain to the inferior vena cava, such as through scrotal vein 146, or via deferential vein 110 to vesicular vein 122. Arterial blood may now enter testes microcirculation 126 unimpeded, allowing recovery of damaged tissues and restoring, at least partially, testosterone production.

In some cases, the pressure in the internal spermatic vein triggers the development of by-pass veins that connect between a lower part of the internal spermatic vein and an upper part thereof or the renal vein. The by-pass veins may restore the excessive pressure on pampiniform plexus 118 and testis 104 with recurrence of at least some of the detrimental effects of excessive high hydrostatic pressure.

In some embodiments of the invention, junction 148 of internal spermatic vein 102 and/or 130 and pampiniform plexus 118 is occluded, preventing the adverse effects of the excessive pressure in the by-pass veins on the venous system of the testis and indirectly on the prostate as described above, while preventing the recurrence of the excessive pressure even if new by-pass veins are developed.

In some embodiments of the invention, junction 148 is occluded with a fast setting sclerosing agent. Optionally, the agent comprises slow flowing or viscous material or a material that gels and/or foams.

In some embodiments of the invention, using a fast setting and/or viscous material prevents the agent from spreading to and occluding other locations, such as pampiniform plexus 118, which may result in various adverse effects.

Optionally, flowing of the sclerosant at least into nearby side-branches is encouraged, to seal such by-pass veins.

In some embodiments of the invention, the deferential vein and/or the vesicular plexus is occluded using microsurgery, optionally by exposing the deferential vein or the vesicular plexus. Optionally, the surgery is conducted under ultrasound or other imaging guidance such as illumination by optical fiber. Optionally, other veins are treated during the operation.

In some embodiments of the invention, the occlusion is carried out by applying sclerosants (sclerosing agents) into a vein. In some embodiments of the invention, the sclerosant comprises, for example, Sodium tetradecyl sulfate in its various forms, ethyl alcohol (e.g., ethanol) or its derivatives, Onyx™, PVA particles, acrylic microspheres, or any blocking agent of the art. Optionally, the sclerosant is applied via intravenous catheter or catheters. Optionally or alternatively, the sclerosant is applied subcutaneously, such as by a catheter or a syringe. Optionally, other methods of blood vessels blocking are used, such as placement of coils, or other mechanical elements such as silk (optionally coated with sclerosant or other materials) that block the vein lumen and/or induce thrombosis that blocks the vein and typically induces degeneration and permanent occlusion.

In some embodiments of the invention, the occlusion agent is a fast setting (curing) cyanoacrylate based glue, such as cyanoacrylate, N-butyl-2-cyanoacrylate (NBCA) (‘glue’), Histoacryl®, or Glubran™.

In some embodiments of the invention, vein sealing is provided by heating and/or ablation, for example, endovascular ablation such as radiofrequency radiation that heats up the vein, or application of direct heating, is used to damage the vein and/or induce its walls to shrink and/or develop a thrombosis, optionally a complete blocking of the vessel. Optionally, a friction against the vessel endothelium may be used to shrink and/or occlude the vessel. Optionally, such treatment, causes an inflammation reaction due to tissue damage, which enhances fiber formation. Optionally, electrocautery such as by electric wire in a catheter, or laser heating by an optic fiber in the catheter may be used to heat and shrink the vessel and/or otherwise effect sclerosis. Optionally, direct electrification is used to occlude a vein. Optionally, cryogenic (hypothermia, freezing) occlusion is used. In an exemplary embodiment of the invention, the catheter (or other apparatus as described herein) include a connector to a source of ablating material/energy. Optionally or alternatively, a control knob or switch is provided on the catheter.

Optionally, these methods are applied by minimally invasive methods such as by laparoscopy. Optionally, the methods are applied externally such as by or radiation, for example, a plurality of laser beams is used to focus at the sclerosis region, while each beam does not damage, or negligibly damage, the other tissues whereas the convergent beams at the focus have sufficient power to shrink and/or effect sclerosis of the vein. Similarly, electromagnetic radiation (e.g. x-ray or by MRI) or ultrasound from several directions focusing at the sclerosis region may be used. Optionally, other mechanical, biological, chemical or physical methods and/or mechanisms, or a combination of said methods and mechanism, may be used to block the blood vessel. Optionally or additionally, a temporary embolization such as by Gelfoam™ (dried gelatin sponge) which clots the vessel and later on dissolves may be used, alone or I conjunction with other methods. Various vessel sealing techniques as known in the art may be adapted for the sizes and/or access as described herein and used. Optionally, the sealing is immediate. Optionally or alternatively, the sealing takes several minutes.

In some embodiments of the invention, a sclerosant is used for the manufacture of a medicament for affecting drainage veins in the groin area and/or forestalling and/or treating BPH or prostate cancer in a subject. Optionally, the sclerosant is adapted to treating backflow that effects BPH and/or prostate cancer. Optionally, the adaptation comprises the composition of materials and/or their proportions, for example, mixing two or more occlusion materials, optionally comprising temporary occlusion material such as Gelfoam™. Optionally and additionally, the medicament may comprise materials with affinity to testosterone and/or adapted to bind to and occlude vessels containing high concentration of bound and/or free testosterone. Optionally, the high concentration comprises 5 to 10 fold, or higher (e.g. 50 to 100 fold), than the normal range of bound and/or free testosterone. Optionally, the medication is administered systemically or locally, for example to the deferential vein. Optionally, the materials with affinity are cleared away at low testosterone levels but cannot be cleared away fast enough at high levels.

In some embodiments of the invention, an antiandrogen medication such as an antigen bound guided molecular therapy may be used as part of the treatment. Optionally, the antigen reduces testosterone production by affecting regions in the brain (e.g. hypophysis or hypothalamus) that regulate testosterone production. Optionally, the antiandrogen comprises materials such as is LHRH analogs (luteinizing hormone-releasing hormone), administered systemically or as subcutaneous patch. Optionally, such antiandrogen material may be a part of the medicament described above. Optionally, the antiandrogen comprises a-reductase blocker which inhibits the conversion of free testosterone to dehydrotestosterone (DTH) which is about 5 fold more potent androgen than free testosterone. Optionally, such anti-androgen is provided in kit form with the sclerosant and/or other blockage means.

Optionally or additionally, anti-androgenic agent may be administered, locally or systemic, to further the healing effect. Optionally, the additional medication may lower even more the testosterone levels without significantly affecting the patient health. Optionally, chemotherapy, brachytherapy, radiotherapy and/or thermotherapy are used to reduce prostate cancer and/or metastases.

In some embodiments of the invention, deferential vein 110 or vesicular plexus 128 is occluded to block the backflow of testicular venous blood into the prostate, relieving it of the excessive pressure and/or high testosterone, and optionally allowing the prostate to recover, at least partially. Optionally, other veins are not treated for backflow and/or varicocele, at least for a time, according assessment of the subject health and/or according to the medical diagnosis and/or medical prognosis with respect to the subject conditions.

In some embodiments of the invention, the prostate is relieved of the back flow by occluding deferential vein 110 or vesicular plexus 128. When deferential vein 110 is occluded the prostate can drain the excessive blood with high concentration of testosterone via vesicular plexus 128 and vesicular vein 122, whereas when vesicular plexus 128 is occluded the prostate can drain via other veins such as vessels such as vessels developed due to the pressure in the prostate. With the excessive venous pressure in the prostate relieved, arterial blood can more easily enter prostate microcirculation 124. Optionally or additionally, the recovering prostatic tissue, with arterial blood with normal testosterone levels (and bound serum testosterone) can reduce the stimulus to growth of cancer tissues in the prostate (that was affected by high concentration of free testosterone).

In exemplary embodiments of the invention, once the driving force for cancer cells development, i.e. high concentration free testosterone (of DTH), is eliminated from the prostate, the prostate cells are deprived of the stimulus to proliferate, and are converted, at least partially, to normal and/or harmless cells and/or may die back.

In some embodiments of the invention, only deferential vein 110 or vesicular plexus 128 are occluded, particularly but not limited to, in case of metastases or suspicion for metastases. The occlusion of such veins allows the prostate to drain, for example via vesicular vein 114 or other veins (such as developed veins due to the pressure in the prostate), relieving the prostate of the pressure and high concentration of testosterone, and allowing the prostate to heal, at least partly, as the cancer cells recover in the absence of free testosterone (or normal ranges of free testosterone). Since testosterone generation and/or drainage to the bloodstream via internal spermatic veins 102/130 is inhibited by the excessive pressure EP, testosterone production and supply to the bloodstream is reduced, reducing the risk for metastases proliferation by high concentration of testosterone, and particularly of free testosterone.

In some embodiments of the invention, the occlusion treatments are useful in forestalling prostate cancer metastases by either (a) occlusion as described above, preventing the development of cancer, and hence, metastases, or (b) if BPH or cancer is already present, occlusion (e.g. by microsurgery) of at least the deferential vein or other vessels that drain from the prostate to the blood stream, possibly allowing the prostate to heal (at least partly) as described above. The occlusion blocks at least some of the venous passage from the prostate and consequently reduces possible leakage of cancerous cells from the prostate that may settle at certain organs and, additionally, may reduce testosterone supply to the bloodstream, reducing the risk of proliferation of metastases.

Possibly, the various effects in the prostate reverse at different rates. For example, hypertrophy caused by excessive pressure may reverse relatively quickly, for example, within a few days or weeks, while hyperplasia due to proliferation, may reverse over a time period of months. For example, in a study by the inventors on 35 patients with BPH, PSA went down a noticeable amount (3.9->3.5) only after 6 months. In an exemplary embodiment of the invention, prostate enlargement caused by pressure is distinguished from enlargement caused by proliferation by manipulation of the prostate to feel its texture and/or elasticity and/or by tracking reduction in volume and/or PSA over time. If the prostate stops shrinking after a few weeks, PSA does not go down and/or prostate is stiff in spite of drainage correction (e.g., volume may go down, but not close to normal), this may indicate androgen-insensitive proliferation. This may suggest immediate biopsy and/or resection and/or irradiation (or other treatment) of prostate despite lack of clinical symptoms. In alternative cases, a reversal of shrinkage and/or reoccurrence of varicocele indicates a failed procedure.

### Exemplary Treatment for Localized Prostate Cancer

In some embodiments of the invention, when prostate localized cancer is detected determined or suspected (e.g. by PSA level and/or biopsy), and metastases are not detected (e.g. by radiology or nuclear imaging methods), an occlusion of a vein connecting the prostate to the pampiniform plexus, such as the deferential vein or the vesicular plexus is indicated.

In some embodiments of the invention, the occlusion is accompanied by antiandrogen administration. Optionally, antiandrogen treatment is not used, for example, when the patient develops, or suspected to develop, adverse effects or symptoms.

In some embodiments of the invention, the occlusion relieves the prostate as described above.

In exemplary embodiments of the invention, antiandrogen medication is optionally applied to further reduce testosterone reaching the prostate. Optionally, the antiandrogen medication is administered, at least partially, locally to the prostate and/or at least a part of the prostate environment such as vessels connecting to the prostate.

In some embodiments of the invention, the antiandrogen medication is applied after a delay from the occlusion, for example, a week after the occlusion of the deferential vein or the vesicular plexus. Optionally, the delay is indicated according to surveillance of the patient condition and/or determination of the treatment effect.

In some embodiments of the invention, the treatment further comprises other medications, for example, chemotherapy. Optionally, radiotherapy and/or brachytherapy and/or thermotherapy and/or cryotherapy (cryogenic therapy) are also used.

### Exemplary Treatment of Metastatic Cancer

In some embodiments of the invention, when prostate cancer metastases are detected or determined (e.g. by nuclear imaging methods such as PET or SPECT), an occlusion of a vein between the prostate and pampiniform plexus is indicated:

In some embodiments of the invention, the vein is one of the deferential vein or the vesicular vein.

In some embodiments of the invention, the internal spermatic vein is not occluded, so that the testis optionally remain in a state of lower testosterone production (relative to normal conditions) due to the excessive hydrostatic pressure, and the metastases are nourished with a smaller amount of serum testosterone relative to normal conditions. Possibly, the low concentration of testosterone causes at least some of the metastases cells to die and/or to convert to harmless cells.

In exemplary embodiments of the invention, antiandrogen medication is optionally applied to further reduce testosterone in the blood circulation. Optionally, antiandrogen treatment is not used, for example, when the patient develops, or suspected to develop, adverse effects or symptoms.

In some embodiments of the invention, the antiandrogen medication is applied after a delay from the occlusion. Optionally, the delay is indicated according to surveillance of the patient condition and/or determination of the treatment effect.

In some embodiments of the invention, the treatment further comprises other medications, for example, chemotherapy. Optionally, radiotherapy is used for metastases that are detected or suspected in a determined region of the body

In exemplary embodiments of the invention, the metastases cells developed in a prostate having a high concentration of testosterone due to reflux, as described above. Consequently, in some cases, such cells need a high concentration of testosterone for survival, and occluding only the deferential vein or the vesicular plexus and leaving the tested in reduced testosterone production by not occluding the internal spermatic veins can enhance the therapeutic effect for such cells by depriving them of normal testosterone. Optionally, the antiandrogen further reduces the testosterone supply to the cancer cells, further depriving them from the concentration of testosterone needed for their survival.

Optionally or alternatively, an anti-androgen releasing implant is provided in the differential vein and/or venous plexus, with or without occlusion.

### Exemplary Treatment Procedures Including Drugs for Localized Prostate Cancer

For localized Prostate cancer there are generally 3 defined stages

(i) Low risk prostate cancer defined when Gleason score≦6 and PSA≦8

(II) Intermediate risk prostate cancer defined when Gleason score=7 and PSA≦20

(iii) High risk prostate cancer defined when Gleason score≧8 and PSA≧20

In an exemplary embodiment of the invention, for Low risk prostate cancer, the following protocol is used:

1. Treatment for Bilateral Varicocele (for example as described herein).

2. 5-6 months later—prostate biopsy.

3. If Biopsy still positive then one injection of GnRH agonist (Goseralin or Buseralin) or GnRH antagonist. (Androgen Deprivation)

4. Follow-up by ultrasound every month for a year.

5. PSA follow-up, every 3 months.

In an exemplary embodiment of the invention, for Intermediate risk prostate cancer, the following protocol is used:

1. Treatment for Bilateral Varicocele.

2. At the same time as the treatment of the varicocele, one injection of Goseralin or Buseralin once a month for 3 months.

3. Follow-up every 3 months by ultrasound for a year.

4. Follow-up after 6 months by prostate biopsy.

5. PSA follow-up, every 2 months for 2 years.

In an exemplary embodiment of the invention, for high risk prostate cancer, the following protocol is used:

1. Treatment for Bilateral Varicocele.

2. At the same time as the treatment of the varicocele, one monthly injection of Goseralin or Buseralin for 6-12 months.

3. PSA follow-up, every 2 months for 2 years

4. Ultrasound follow-up every one months

5. Prostate biopsy after 6 months.

In an exemplary embodiment of the invention, for prostate cancer with metastases, the following protocol is used:

1. Occlusion of the DV only, for both sides.

2. At the same time as the treatment of DV, apply a 6-12 month Androgen Deprivation Therapy by Goseralin or Buseralin injections.

3. PSA follow-up, every 1 month for 2 years.

4. Ultrasound follow-up every 1 month.

5. Prostate biopsy after 6 months.

### Exemplary Chemotherapy for Prostate Cancer

Following are examples of medication and dosages which may be used for treating prostate cancer. A first class of medication is AAT (ADT), LHRH agonists. Examples include:

(a) Zoladex (injection), for example, 10.8 mg every 3 months.

(b) Lucrin (injection), for example, 11.25 mg every 3 months.

(c) Superfact depot (injection), for example, 9.9 mg every 3 months.

In an exemplary embodiment of the invention, such medications are provided in conjunction with mechanical reduction of testosterone flow to the prostate. Optionally, the dosages used are smaller and/or less frequent, for example, being applied at 80%, 70%, 50%, 30%, 20% 10% or intermediate dosages. Optionally, the frequency is 70%, 50%, 40%, 20% or other frequency ratios. Optionally, the frequency is made higher with dosages smaller, to allow better control over testosterone levels.

A second class of medication is Androgen Receptor antagonists. Examples include:

(a) Casodex (Biclutamide), for example at 50 mg x1/day, if provided with ADT (androgen deprivation therapy) and 150 mg x1/day if provided without ADT.

(b) Flutamide, for example at 250 mg x3/day

(c) Ciproterone acetate (Armoeur), for example at 100 mg x2/day

A third class of medications is Estrogen, for example, DES —diethyl stilbestrol, for example, 1×gr/day for a week then 0.5 gr/day twice a week

A fourth class of medication is Anti adrenal Testosterone production, including, for example:

(a) Ketokanazole 2×200 mg/d

(b) Abiroteron, in development, possible dosage at 1000 mg Abiroteron-Actetat together with 2×5 mg/d Prednisolon.

A fifth class of medication is a combination therapy of Total Androgen Ablation (TAB/CAB), for example, LHRH plus an Antiandrogen.

A sixth class of medication Blockades the conversion of Testosterone to DHT, for example, an alfa reductase inhibitor (Fenasteride), for example, at 5 mg/d

A seventh class of medication is Chemotherapy, for example, Daxotrene (Docetaxel), for example, one injection/3 weeks.

### Exemplary Medication Targets

In general, the various medications (other than chemotherapy) are used to reduce testosterone existence and/or acceptance by prostatic cancer cells, and thereby causing the reversion and/or deterioration of hormone-dependent cancer cells. However, it has been surprising realized by the inventors of the present application that this goal cannot generally be achieved, even by drastic reduction of serum testosterone (e.g., medical castration), as long as venous black flow (which may initially cause the disease), still exists. This has been also borne out by some researchers (e.g., Elahe A. Mostaghell, Stephanie T. Page, Daniel W. Lin, Ladan Fazli, Ilsa M. Coleman 1, Lawrence D. True, Beatrice Knudsen, David L. Hess, Colleen C. Nelson, Alvin M. Matsumoto, William J. Bremner, Martin E. Gleave and Peter S. Nelson, Intraprostatic Androgens and Androgen-Regulated Gene Expression Persist after Testosterone Suppression: Therapeutic Implications for Castration-Resistant Prostate Cancer. Cancer Research 67, 5033-5041, May 15, 2007), which found that after androgen ablation therapy (AAT), intra-prostatic testosterone levels are higher than serum levels. While the serum level reduced to 4-5% or normal, intra-prostatic levels decreased to 20-30% of normal only which means that intra-prostatic remain some six fold higher than expected. It is suggested by the present inventors that this is caused by the venous backflow.

Rather, most testosterone appears to reach the prostate via the above mentioned venous system, which not only provides testosterone which is not affect by flow through the body, but may also provide free testosterone at very high levels (as blood flow time is not sufficient to cause protein-binding thereof). Thus, if the venous flow provides up to 130 times the amount of free testosterone to the prostate than provided via an arterial serum route, even if the serum level is depressed by 95%, the level reaching the prostate will still be ˜6 times the normal free testosterone level.

Additionally, the inventors hypothesize that anti-androgen treatment have a detrimental effect in that they may encourage cancer proliferation. When anti-androgen treatment is provided, the testosterone levels in the prostate go down, but are still some 6 fold above normal. This means that while some cancer cells may die, others have the opportunity to evolve, possibly into forms that are less sensitive to testosterone and/or do not require such testosterone. Possible, this is caused by mutation or by another selective pressure, for example, one which allows previously less-competitive cells (those requiring and using less testosterone) to become competitive. These are theories and the following should not be necessarily limited to these explanations.

If, in a previous disease stage, prostatic cancer cells could not survive outside the prostate, due to their high-testosterone needs, once the cancer evolves, such cells can survive and metastasize. Moreover, once a cancer has reached a state where some cancer cells can live outside the prostate, this may indicate a sizable number of cells that are not super sensitive to hormone levels, inside the prostate.

Additional detrimental effects of anti-androgen treatment exist, including a negative effect on the heart, bones (osteoporosis) flexibility of muscles and tendons, immune system, memory, concentration, depression, decreased libido and/or fatigue, due to testosterone levels that are below maintenance levels.

In an exemplary embodiment of the invention, the above or other venous blockage methods are used to prevent direct and abnormal testosterone flow from the testis to the prostate. In an exemplary embodiment of the invention, such blockage is performed before any anti-androgen treatment, to prevent the possibility that prostatic cells will be exposed to moderately high testosterone or above-minimal levels (caused by AAT) which might enhance their evolution. Rather, androgen levels are immediately brought down to normal (or below normal). In an exemplary embodiment of the invention, the level to which androgens are brought is selected so that it will have a shock effect and will have an anti-proliferative effect on prostatic cancer cells and/or pre-cancerous cells. Optionally, a combination of venous blockage and serum reduction is used (e.g., to compensate or overcompensate for serum increase due to treatment of varicocelle). Optionally, a venous blockage method that does not increase testosterone production is used.

In an exemplary embodiment of the invention, what is desired is to bring testosterone levels in the serum to be within a normal range, while simultaneously reducing prostatic levels to normal. In some cases, both serum and prostatic levels are reduced to below normal. In some cases, by bringing both serum and prostatic levels to near normal, a double positive effect is achieved, normal serum levels enhance body health, while normal prostate levels cause cancer regression. In some embodiments of the invention, the amount of anti-androgens used are smaller than in art, as it is desired to maintain a higher serum level than was required in art. In some embodiments, both serum and prostate levels are below-normal, the prostate levels set to cause healing and the serum levels set to avoid damage to body.

Complete androgen block (CAB) (ablation of testosterone production in the testes and adrenal glands) if used, can be, for example, to target the testis and adrenal glands by ketocanazole.

In some embodiments of the invention, multiple levels of prostatic testosterone are provided selectively, for example, by mechanical venous blockage and/or varying amounts of anti-androgen treatment. For example, while prior practice may have been forced to reach a very low serum androgen level to have an effect, methods in accordance with exemplary embodiments of the invention can provide effective prostatic cancer suppression at multiple serum levels. Optionally, a protocol is provided where several serum levels are tried (e.g., to assess general health at the levels), while ensuring a below-minimal prostate level is achieved for all such levels.

In an exemplary embodiment of the invention, an attempt is made to ensure prostate hormone levels remain high without androgen deprivation therapy (ADT), until the suggested treatment and do not dip for long periods of time, to prevent the emergence of low-testosterone sensitivity cancer cells, before cancer treatment is started.

In an exemplary embodiment of the invention, once it is estimated that such low-sensitivity cells are already in existence, the prostate is removed, as various anti-androgen therapies may be less effective. Optionally, after prostatectomy in case of, metastases treatment of CAB can be given.

### Feedback

In an exemplary embodiment of the invention, the treatment includes a feedback process. In one example, feedback includes performing a biopsy of the prostate to measure androgen levels in situ. Optionally or alternatively, feedback includes estimating prostate hormone levels based on a known venous backflow state and serum levels. Optionally or alternatively, feedback includes selecting dosage levels and treatment protocols according to a known state of venous backflow, for example, to provide a different treatment to a cancer patient with no venous backflow. Optionally or alternatively, feedback includes imaging and estimating testosterone levels in the prostate, for example, by mapping venous backflow (e.g., by injection of contrast material into the ISV or other veins) and viewing backflow using x-ray. Optionally or alternatively, radioactively tagged testosterone is injected into the ISV or other location and its arrival at prostate imaged using a scintillator-imager or sensor.

It is a particular feature of some embodiments of the invention that prostatic hormone levels can be substantially reliably estimated from serum levels. Optionally or alternatively, the prostate is biopsied and an estimate of free testosterone, bound testosterone and/or DHT are made based on concentration per tissue unit biopsied.

In an exemplary embodiment of the invention, feedback on the type of tissue in the prostate is estimated base don the response of the prostate to treatment, for example, venous reflux blockage. For example, it is expected that mechanical shrinkage take effect substantially immediately and complete within a few weeks or months. Biological shrinkage can take longer. For example, PSA levels go down for six months or more after BPH is treated. Optionally, if shrinkage does not continue and/or PSA levels do not go down and/or prostate does not enlarge after AAT is stopped and/or after backflow to testis is stopped, androgen-insensitive tissue is assumed to exist and resection and/or other aggressive treatment may be applied to the prostate. Optionally or alternatively, if varicocele is seen to exist or recur, it is treated again. Optionally, a delay until the veins expand sufficiently to be mapped and/or passed with a catheter is waited, for example, several months. During such time, AAT may be applied or avoided, for example, as discussed herein.

In an exemplary embodiment of the invention, even after venous treatment, a repeated checks for varicocele are carried out, for example, every few months and/or years, optionally with decreasing frequency, e.g., at 1 month, 3 month, 6 month, 1 year, 3 years. Optionally, if AAT is provided, such checking is carried out during treatment.

Exemplary Target Levels

In an exemplary embodiment of the invention, any of the abnormal parameter values is used as a target for correction during treatment by venous blockage and/or medical treatment. Optionally or alternatively, a plurality of parameters are selected and target ranges selected for each. For example, two, three, four or more parameters may be selected and various parameters of the treatment (e.g., degree of blockage, location of blockage, type of medication, dosage and/or frequency) are modified using various optimization and control methods, for example, those known in the art of medicating, to achieve or approximate the desired ranges. In some cases, for example for androgen, a value indicating a specific androgen activity is used instead of androgen levels, for example, combining the effects of bound testosterone, free testosterone and DHT.

For example, target values may be set to be within normal range (e.g., +−20%). In some cases, within a time frame of several weeks or several months normal values are not expected (e.g., for prostate volume). Target values may be, for example, for elevated parameters, 300% of normal, 200% of normal, 150% of normal 120% of normal 100% of normal, 80% of normal, 50% of normal or intermediate or smaller or greater values. For testosterone levels which indicate an subnormal testosterone level, range sin prostate and/or serum may be, for example, as described below, generally, “normal”, “Sub-normal”, “low” (˜5%) or very low (˜0%). As noted above, it is a particular feature of some embodiments of the invention that serum and prostate levels are linked differently after venous blockage than without such blockage and during partial or complete reflux. In some cases, there are additional drainage pathways for testis, so not all testicular output goes to prostate. Ratio can be assessed, for example, by injecting contrast material into the testicular vein or artery and tracking its spread I the body using X-ray.

In an exemplary embodiment of the invention, it is desired to achieve pressure levels in the prostatic drainage of less than 300%, 200%, 150%, 120% of normal values. Optionally, the above percentages are of a nominal value, such as 5 mmHg.

In an exemplary embodiment of the invention, it is desired to reach a target prostate volume, for example, 25 ml, 30 ml, 40 ml, 50 ml or intermediate sizes, or a reduction of, for example, 20%, 30%, 40%, 50% or intermediate values, within 1 month, 2 months, 3 months, 6 months or intermediate values.

In an exemplary embodiment of the invention, it is desired to achieve a serum (and thereby prostate) serum level of about 8.4 nmol/ml. Optionally, the target level is lower, for example, 1, 2, 3, 4, 5 nmol/ml or intermediate or lower serum levels. Optionally or alternatively, higher than normal levels are desired, for example, 10, 13, 20, 30 nmol/ml or intermediate or higher values. Normal levels might depend on the age of the patient.

In an exemplary embodiment of the invention, prostate androgen levels are estimated based on biopsies which show prostate testosterone, free-testosterone and/or DHT levels as a function of volume, typically measured in nanograms. Optionally, the desired levels are less than 200%, 100%, 80%, 50%, 30%, 20% of normal levels. Optionally, what is looked for is a gradual reduction in such values.

In an exemplary embodiment of the invention, the treatment includes setting a base line and setting an allowed variation, for example, 10%, 20%, 30%, 40%, 50% or intermediate or greater amounts, and tracking the serum levels, for example, daily, bi-daily, weekly, bi-weekly or at other higher or lower frequencies to adjust an anti-androgen treatment. Optionally, AAT is provided at a lower dosage and higher frequency to better track such measurements and avoid too low and too high serum and/or prostate androgen levels.

In some cases, feedback and target values as described herein are used for treating prostate cancer with only venous blockage treatment and/or only treatment without venous blockage. Optionally or alternatively, it is used for combined treatment including vein blocking and one or more of chemotherapy, radiation, cryoablation, heating and/or focused ultrasound.

### Exemplary New Regimens

An exemplary regimen for high risk PCA (e.g, Gleason 8,9,10), without metastases is as follows:

(a) Check for bilateral Varicocele. It is noted that also sub-clinical manifstations and/or symptomless manifestations may be identified and treated in this and other embodiments of the invention.

(b) treat Varicocele and/or otherwise block venous backflow to prostate.

(c) Examine Prostate volume regularly (e.g., weekly) by ultrasound or other means. If size of prostate persistently decreases in response to varicocele treatment, then apply anti-androgen treatment for at least 6 months (e.g., 2 injections), optionally with a target level of 5% of normal serum testosterone levels. Optionally, higher levels are allowed, for example, between 30% and 70%. It should be noted that if varicocele is treated (as opposed to only blocking venous backflow to prostate) the dosages required to reach a “standard” serum level may be higher, due to increased health of testis.

An exemplary regimen for Metastatic Prostate cancer without previous anti-androgen treatment, is as follows:

(a) block venous backflow to prostate

(b) start CAB immediately for 6 months.

An additional exemplary regimen for Metastatic Prostate cancer without previous anti-androgen treatment, is as follows:

(a) block venous backflow to prostate.

(b) Radical prostatectomy and irradiation, simultaneously and/or after a delay.

(c) start CAB immediately for 6 months.

The above are only exemplary regimens and the duration of treatment could be modified. Optionally or alternatively, additional treatments are performed, for example, chemotherapy and injection of an anti-androgen into the prostate, for example, in the form of a pharmaceutical eluting implant. Optionally, an alpha-reductase inhibitor is implanted in or injected into the prostate.

In an exemplary embodiment of the invention, if previous AAT was applied before blocking/preventing of venous reflux, a stronger AAT is used, for example, down to 4%-%% or even lower, such as 3%, 2%, 1% or less.

In an exemplary embodiment of the invention, CAB is applied closer to toxic concentrations, for example, ketocanazole 200×4/d.

In an exemplary regimen for localized prostate cancer, after venous reflux prevention, a single shot of AAT is provided, for example, a single injection of GNRH analog, that has an effect for ˜3 months rather than 6 months. Optionally, if a biopsy shows maintenance of cancerous and/or pre-cancerous cells, an additional shot is given. Optionally or alternatively, the dosage is reduced (e.g., by 60%, 50%, 40% or intermediate or greater amounts) and applied more frequently (e.g., taking into account the half life of injected medication) thus allowing a higher serum level of androgen to be maintained. Optionally, a vacation is provided between multiple applications, to allow the body to recover.

### Exemplary Preventive Treatment for Prostate Disorders

In some embodiments of the invention, a preventive treatment is applied to a subject in order to prevent the development of prostate disorders such as BPH and cancer.

In exemplary embodiments of the invention, subjects are screened for prostate disorder risks or for early stages of prostate disorders. Optionally, the screening comprises detection or determination of uni-lateral (left or right side) or bi-lateral (left and right side) varicocele of the internal spermatic veins and/or testicular veins.

In some embodiments of the invention, the risks are assessed by measuring the testis temperature, wherein elevated temperature optionally indicates varicocele. Optionally, the external temperature of the testicular sack is measured. Optionally, a thermograph is used for external measurement of the testes temperature.

In exemplary embodiments of the invention, a temperature over 32° C. is an indication of a varicocele, such as 32.5° C. or above. Optionally, a temperature over 34° C. is an indication of a varicocele. Optionally, a temperature over 36° C. is an indication of a varicocele. Optionally, a temperature over 37° C. is an indication of a varicocele. Optionally, a temperature between the indicated temperatures is an indication of a varicocele.

In some embodiments of the invention, concentration of PSA and/or other markers are used to assess prostate disorder risks or early stages of prostate disorders.

In some embodiments of the invention, venography and/or ultrasound is used to determine or assess varicocele and/or venous anatomy and/or prostate anatomy. For example, an enlarged and/or congested prostate may indicate a risk for prostate disorders, or distended veins may indicate varicocele.

In exemplary embodiments of the invention, the preventive treatment comprises occlusion of the internal spermatic vein 102 and/or right internal spermatic vein 130, so that that the excessive hydrostatic pressure developed in the varicose internal spermatic veins is blocked. The reflux of high pressure venous blood rich in testosterone is prevented from reaching the prostate, enabling a congested prostate and/or a prostate with hyperplasia to recover as described above.

In exemplary embodiments of the invention, the preventive treatment comprises occlusion of junction 148, left internal spermatic vein 102, pampiniform plexus 118 and/or junction 148 of right internal spermatic vein 130 and pampiniform plexus 118. Occluding the junction may be advantageous by preventive the recurrence of excessive hydrostatic pressure in the pampiniform plexus (and resultant effects) due to the development of by-pass veins that connect between a low part of the internal spermatic vein and an upper part of the vein or the renal vein.

Optionally, the junction 148 is occluded with fast drying agent such cyanoacrylate based glue as described above.

In an exemplary embodiment of the invention, once a person is found to have BPH, even with a low Gleason score, anti-androgen therapy is provided, for example, for between 1-10 months for example, for 6 months, to ensure that no pre-cancerous cell and/or abnormal cells that may have been encouraged by the high-testosterone state, remain. In some cases, such anti-androgen therapy is applied even if no prostate enlargement is found, for example, if varicocele has existed for a considerable period of time, such as 1 year, 5 years, 10 years or more.

### Occlusion of the Deferential Vein

In some embodiments of the invention, occlusion of the deferential vein is indicated in case of prostate cancer metastasis.

In some embodiments of the invention, the occlusion of the deferential vein disconnects the prostate venous system from the excessive pressure at the pampiniform plexus and/or internal spermatic vein, such that:

(a) the testicular venous blood rich in testosterone is impeded from reaching the prostate, thus maintaining normal blood pressures and testosterone concentration in the prostate, reducing the probability of developing disorders in a substantially healthy prostate; and

(b) prostate venous blood can drain via the vesicular plexus up towards the inferior vena cava, relieving the prostate of the excessive pressure and high testosterone concentration, allowing an ailing prostate to recover, at least partially, as described above.

In some embodiments of the invention, occluding only the deferential vein rather than, additionally or separately, occluding the internal spermatic vein may have advantages for one or more of the following reasons:

(a) elimination the introduction of a catheter or wire in the internal spermatic vein against the flow direction of the one-way valves in the internal spermatic vein;

(b) the deferential vein can be reached via the femoral vein and iliac veins to the vesicular vein relatively easily with respect to reaching up the abdomen and down the internal spermatic vein;

(c) the deferential vein can be accessed subcutaneously; and

(d) the testicular venous system is substantially not affected, optionally maintaining the reduced testosterone production, optionally depriving metastases cells of testosterone for their existence and proliferation.

In some embodiments of the invention, the internal spermatic vein or other veins are treated separately or in a later time, for example, according to a determination of the patient condition.

The deferential vein is a thin-walled narrow and delicate vein normally with a width of about 0.1-0.2 mm, and under the elevated pressure and backflow the vein may expand to a width of about 0.3-0.5 mm.

Additionally, the deferential vein is typically attached to the vas deferens, also a thin and narrow-walled delicate vessel.

In some embodiments of the invention, an opening of the deferential vein is occluded. In such a case, optionally, care should be exercised to avoid occlusion of other veins, such as the cremasteric vein.

In some embodiments of the invention, treating the deferential vein may require care to avoid a damage to the vein or other veins or the vas deferens, and may require special equipment for occlusion.

In some embodiments of the invention, the deferential vein is occluded with the apparatus described below.

### Occlusion of the Vesicular Plexus

Vesicular plexus 128 has one or more branches. Typically, all the branches have to be occluded in order to impede the reflux from pampiniform plexus 118.

In exemplary embodiments of the invention, vesicular plexus 128 branches are occluded as described above, optionally, with the apparatus described below.

The vesicular plexus is not attached to the vas deferens, which may be advantageous, yet all the branches of the vesicular plexus have to be occluded which may prolong and/or complicate the treatment.

### Occluding a Vein Opening

As discussed above, a prostate treatment may require occluding a vein between pampiniform plexus 118 and prostate 124, such as deferential vein 110 or vesicular plexus 128.

The anatomy of the thin and delicate deferential vein 110, particularly as it is attached to the vas deference, may prohibit or present difficulties to insert a catheter into the vein. The anatomy of vesicular plexus 128 is somewhat less demanding but in many cases requires accessing a plurality of vessels that connect to vesicular vein 112.

In some embodiments of the invention, a vein is occluded at its opening, such as the opening of deferential vein 110 or the vesicular plexus 128 into vesicular vein 112, or the opening of deferential vein 110 into pampiniform plexus 118.

In occlusion of the opening of a vein, the sclerosant is optionally applied only to the vein or its opening, excluding access of the sclerosant to other regions, that is, preventing the sclerosant from flowing in vesicular vein 112 or pampiniform plexus 118.

In some embodiments of the invention, a special catheter is used which is particularly adapted to perform occlusion of a vein opening only.

For clarity and brevity in the following discussion, without limiting generality, deferential vein 422 (110) is referred to as an example of a vein branching from another vein and vesicular vein 420 (112) is referred to as an example of a vein into which a branching vein opens.

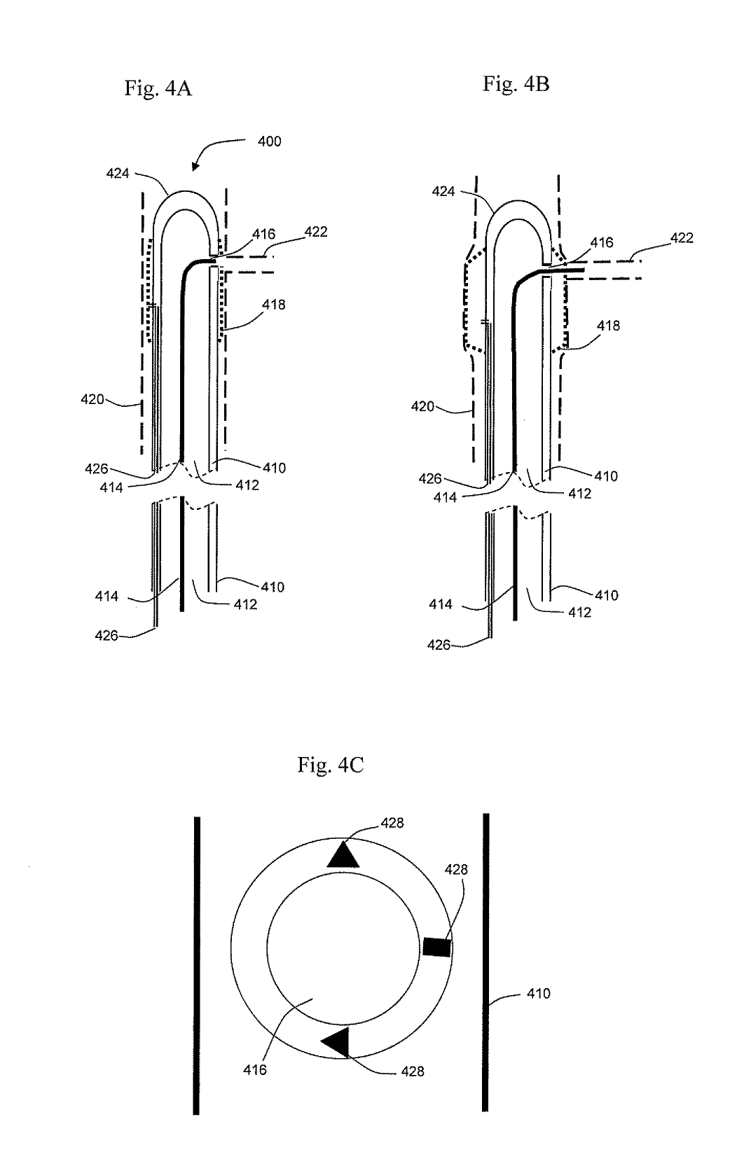

FIG. 4A schematically illustrates a catheter 400 designed to align with an opening of the deferential vein and to inject a sclerosing agent into the deferential vein, while preventing the agent to reach other regions of the vesicular vein by an expandable element shown in collapsed state, in accordance with some embodiments of the invention. FIG. 4B illustrates catheter 400 showing the expandable element in expanded state, in accordance with some embodiments of the invention.

In some embodiments of the invention, catheter 400 is configured to apply a sclerosant at an opening of the deferential vein of a width of about or less than about 0.5 mm, such as about 0.4 mm or about 0.3 mm. In some embodiments of the invention, catheter 400 comprises a tube 410 having a lumen 412 with an orifice 416 adapted to adhere to and inject a sclerosant to the opening of the deferential vein. Optionally, orifice 416 is located at the side of tube 410 near the distal end (towards the body) of catheter 400. While in some embodiments, the orifice is aimed to be substantially perpendicular to a long axis of the catheter, in some embodiments, it is at various angles. Optionally, the angle is not fixed and is varied, for example, by manipulating (e.g., axially, radially and/or rotationally) an inner tube which encloses lumen 412 attached to the orifice

In some embodiments of the invention, the width of orifice 416 is about 0.5 mm. Optionally, the width is larger than about 0.5 mm such as about 0.6 mm or about 0.7 mm. Optionally, the width is smaller than about 0.5 mm such as about 0.4 mm or about 0.3 mm or about 0.2 mm.

In some embodiments of the invention, the width of catheter 400 is about 1 mm. Optionally, the width is less than about 1 mm, such as about 0.3 mm or about 0.5 mm or about 0.6 mm or about 0.8 mm. Optionally, the width is larger than about 1 mm, such as about 1.2 mm or about 1.5 mm.

In some embodiments of the invention, the distance of orifice 416 from the distal end of catheter 400 is about 1 mm. Optionally, the distance is larger than about 1 mm, such as about 1.5 mm or about 2 mm or about 3 mm. Optionally, the distance is smaller than about 1 mm.

In some embodiments of the invention, catheter 400 comprises a cover 424 closing lumen 412 at the distal end of catheter 400. Optionally, cover 424 forms, at least partially, an integral part of tube 410.

In some embodiments of the invention, a guide-wire 414 is inserted in lumen 412, reaching orifice 416. Optionally, guide-wire 414 is soft and/or flexible, at least at the distal section, enabling guide-wire 414 to enter orifice 416. Optionally, guide-wire 414 is bent at the distal end, further enabling guide-wire 414 to enter orifice 416.

In some embodiments of the invention, guide-wire 414 is fabricated as a part of catheter 400, where guide-wire 414 is, optionally, removable from catheter 400.

In some embodiments of the invention, catheter 400 comprises at least one expandable element 418 around a section at the distal end of catheter 400, with one element 418 having an orifice aligned with orifice 416. Optionally or additionally, two or more elements 418 are expandable to form a passage in front of orifice 416.

In some embodiments of the invention, expandable element 418 is configured to press against the walls of the vesicular vein 420 so as to block leakage of a sclerosant beyond the opening of the deferential vein 422, upstream and/or downstream.

In some embodiments of the invention, expandable element 418 comprises an inflatable balloon, optionally with a lumen 426 inside tube 410 (or wall thereof) for inflating and deflating balloon element 418.

By way of example, without limiting, it is assumed in the following discussions that catheter 400 was maneuvered into vesicular vein 420 (112) near the opening of differential vein 422 (110), and that the expandable element 418 is an inflatable balloon. An exemplary method of maneuvering a catheter to reach an opening of the deferential vein is described later on.

In some embodiments of the invention, an operation of catheter 400 comprises:

(a) moving and/or rotating catheter 400 while pushing and retracting guide-wire 414 until the distal tip of guide-wire 414 enters the opening of deferential vein 420, as illustrated in FIG. 4B; Optionally, moving catheter 400 while guide wire 414 is slightly extended allows guidewire 414 to catch on the opening;

(b) inflating balloon 418 via lumen 426, securing balloon 418 to the walls of vesicular vein 420 as illustrated in FIG. 4B and/or sealing the side branch from the main vessel;

(c) injecting via catheter 400 and orifice 416 a sclerosant agent into deferential vein 420; and

(d) deflating balloon 418 and removing catheter 400 and guide-wire 414.

In some embodiments of the invention, guide-wire 414 is removed before injecting the sclerosant agent. Optionally, an internal catheter is moved inside catheter 400 along guide-wire 414 for injecting the sclerosant agent. Optionally or alternatively, a blocking element such a foam plug is injected instead of a sclerosant.

In some embodiments of the invention, inflated balloon 418 holds catheter 400 in place such that orifice 416 is facing and attaching to the opening of the deferential vein wherein the inflated balloon blocks the sclerosant from reaching vesicular vein 420 and unfavorably occluding it.

FIG. 4C schematically illustrates an orifice 416 at a distal end of the catheter 400, comprising radio opaque elements 428 around and/or near the orifice, enabling to position the orifice at an opening of a branching vein, in accordance with some embodiments of the invention. Other configurations may be used as well.

In some embodiments of the invention, orifice 416 of catheter 400 is aligned with the opening of deferential vein 422 using the asymmetric arrangement of one or more radio-opaque elements 428 about orifice 416. In some embodiments of the invention, during x-ray imaging the asymmetric arrangement of radio-opaque elements 428 enables to distinguish when orifice 416 is facing the opening of the vein or orifice 416 is opposite the opening. Optionally, elements 418 are used in conjunction with guide-wire 414, to allow faster and/or easier initial positioning.

In some embodiments of the invention, an operation of catheter 400 comprises:

(a) moving and/or rotating catheter 400 while imaging with x-ray the position of orifice 416 with respect to opening of deferential vein 422, wherein vesicular vein 420 and/or deferential vein 422 are optionally injected with contrast medium;

(b) expanding balloon 418 via lumen 426, securing balloon 418 to the walls of vesicular vein 420 as illustrated in FIG. 4B;

(c) injecting a sclerosant agent into the deferential vein 420; and

(d) deflating balloon 418 and removing catheter 400 and optional guide-wire 414.

In some embodiments of the invention, a region about the perimeter of orifice 416 is coated with an adhesive material adapted for temporary contact with the vesicular vein 420. Optionally, the material becomes adhesive responsive to a temperature change (e.g., provision of hot water in catheter 400 or exit form a cool catheter) and/or material (e.g., in blood flow and/or provided in the catheter). Optionally, the material becomes non-adhesive or dissolves responsive to a temperature and/or material. For example, using reversible non-toxic, optionally bio-degradable, sol-gel compound (e.g. similar to methyl cellulose) that will adhere to the vein in the gel form and disconnects in the sol form, wherein the transition is affected by temperature. Optionally, the temperature and/or material are affected by flushing lumen 412 with a fluid.

In some embodiments of the invention, an operation of catheter 400 comprises:

(a) guiding catheter 400 such that orifice 416 contacts about the opening of the deferential vein as described above.

(b) flushing a fluid to affect adhesion of catheter 400 to the walls of the vesicular vein around the opening of the deferential vein such that orifice 416 faces the opening;

(c) injecting sclerosant into the opening of the deferential vein; and

(d) flushing a fluid to affect removal of catheter 400 from the walls of the vesicular vein.

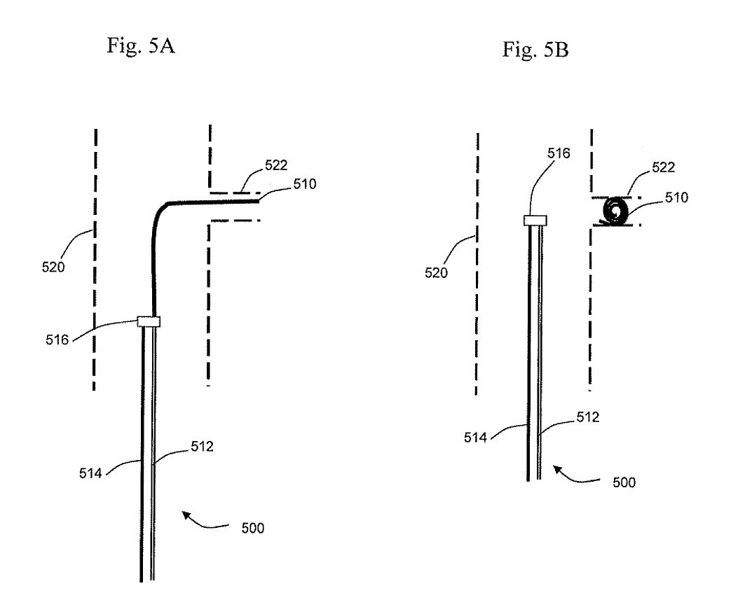

In some embodiments of the invention, catheter 400 can be used to deploy a coil as described below with respect the apparatus of FIG. 5.

In some embodiments of the invention, catheter 400 is configured to access the deferential vein subcutaneously in a procedure similar to laparoscopy.

### Deployable Coil

In some embodiments of the invention, deferential vein 110, or vesicular plexus 128, is occluded by a coil (or spring) deployed in the vein. The occlusion is carried out by the coil body and/or by thrombosis induced by the coil. In some embodiments of the invention, the coil is contractible from a thin element such as a strip.

In some embodiments of the invention, the coil is deployed subcutaneously using procedure similar to laparoscopy. For clarity and brevity in the following discussion, without limiting generality, deferential vein 522 (110) is referred to as an example of a vein branching from another vein and vesicular vein 520 (112) is referred to as an example of a vein into which a branching vein opens.

FIG. 5A schematically illustrates an apparatus 500 for deploying a coil 510 (shown in a stretched state) in the deferential vein (or another branching vein), while FIG. 5B schematically illustrates apparatus 500 after the coil is deployed, in accordance with some embodiments of the invention.

Apparatus 500 and coil 512 are configured and adapted to reach the small opening of the deferential vein of a width of about or less than about 0.5 mm (down to about 0.25 mm or 0.2 mm), and to be inserted into the vein without inflicting damage to the delicate vas deferens that is attached to deferential vein 110.

In some embodiments of the invention, initially coil 510 is formed from a strip. Optionally, the strip width is suitable and/or adapted for insertion at the vein opening and inwards into the vein. Optionally, the strip width is smaller than the vein opening such that it can be maneuvered during the procedure of insertion into the vein. Optionally, the width is about 0.25 mm. Optionally, the width is larger such as about 0.3 mm, about 0.4 mm, about 0.5 mm, or an intermediate value. Optionally, the width is less than about 0.25 mm, such as about 0.2 mm.

In some embodiments of the invention, coil 510 is made of a shape memory alloy (SMA) such as Nitinol™.

In some embodiments of the invention, coil 510 coils from a strip form responsive to temperature in the vein. Optionally or alternatively, coil 510 is coiled responsive to heat, for example, by inducing current with electromagnetic radiation.

In some embodiments of the invention, apparatus 500 comprises:

(a) a wire (and/or tube) 512 adapted to maneuver to the opening of deferential vein 522;

(b) a release mechanism 516 mounted on the distal end (towards the body) of wire 512.

In some embodiments of the invention, release mechanism 512 is mounted with coil 510, in the initial form of a strip, on the distal end of wire 512

In some embodiments of the invention, release mechanism 516 is controlled by a control wire or rod 514.

By way of example, without limiting, it is assumed in the following discussions that apparatus 500 was maneuvered into vesicular vein 520 (112) near the opening of the differential vein 522 (110). The path for maneuvering an apparatus to reach an opening of the deferential vein is described later on.

In some embodiments of the invention, an operation of apparatus 500 comprises:

(a) maneuvering coil 510 (in a strip form) to the opening of deferential vein 522;

(b) inserting coil 510 into deferential vein 522; and

(c) releasing coil 510.

Optionally, scelrosant and/or adhesive are provided as well.

In some embodiments of the invention, apparatus 500 is maneuvered inside a catheter that optionally protects the vessels walls from damage by apparatus 500 and/or optionally facilitates the maneuvers in the veins, such as tube catheter with open distal and proximal ends. Optionally, apparatus 500 is operated after the catheter is pulled back to enable the deployment of coil 510. In some embodiments of the invention, the catheter is used to flush coil 510 and/or release mechanism 516 with liquid in a temperature adapted for maintaining coil 510 and/or release mechanism 516 in a particular fowl suitable for the operation.

In some embodiments of the invention, with reference to FIG. 4, apparatus 500 and coil 510 are inserted in catheter 400. Optionally, coil 510, in a strip form, is inserted via orifice 416 into the opening of deferential vein 522, where the guiding of catheter 400 as described above optionally helps in positioning the end of coil 512 in the strip form at the opening of deferential vein 522. Optionally, the insertion is carried out when catheter 400 is coupled to vesicular vein 520 as described above with reference to FIG. 4. Optionally, lumen 412 of catheter 400 is used to flush a fluid with a temperature suitable to affect the coiling of coil 510.

In some embodiments of the invention, release mechanism 516 is as known in the art of stent or other grafts deployments. For example, coil 510 is held by a latch (e.g. a pin) that is released by pulling control wire 514. In another example, release mechanism comprises a shape memory element configured to release coil 510 and is released when subject to a suitable temperature, such as by flushing it with a fluid in an optional catheter around apparatus 500 and/or upon reaching body temperature.

Subcutaneous Looping and/or Coagulation

In some embodiments of the invention, deferential vein 110 or vesicular plexus 128 is occluded by a procedure and equipment similar to laparoscopy and performed from outside the blood vessel.

In some embodiments of the invention, the occlusion comprises an electric coagulation applied to the external walls of the vein. Optionally, the occlusion is effected by heat produced by electric current. Optionally or alternatively, the coagulation is effected by hot fluid. Optionally or alternatively, the coagulation is effected by cold fluid.

Typically deferential vein 110 is attached to the vas deferens, both being delicate vessels, in the order of sub-millimeter width, such as about 0.2 to about 0.5 mm, and typically vesicular plexus 128 has a plurality of branches.

In some embodiments of the invention, in order to occlude deferential vein 110, the vein is separated from the vas deferens in order not to damage the latter. Similarly, each branch of the plurality of branches of vesicular plexus 128 is optionally separated from the surrounding tissues.

In an exemplary embodiment of the invention, an apparatus is provided which performs a combined separation and occlusion. In other embodiments, coagulating energy is provided from outside the vein, on opposite side from sensitive tissue.

For clarity and brevity in the following discussion, without limiting generality, deferential vein 610 (110) is referred to as an example of a vein branching from another vein and vesicular vein 112 is referred to as an example of a vein into which a branching vein opens, whereas the a vas deferens 612 represents also tissues around the vesicular plexus.

In the following discussion, the term ‘spike’ denotes an elongated apparatus adapted to separate a vein from the surrounding tissues and to form a loop at one end around the vein.

Additionally, in the specifications and claims, the term ‘loop’ denotes a closed loop or, optionally or alternatively, an arc optionally nearing a closed loop form, such as about 270° or more such as about 300° or about 360° or more. In some embodiments, a partial loop, for example, of 90°, 120°, 150° or intermediate or greater angle may be used.

To clarify the following procedure, it may be optionally summarized as follows:

(a) apply imaging (e.g. optical, ultra sound, x-ray);

(b) introduce the spike into the abdomen, under imaging guidance;

(c) separate at least a part of the deferential vein (or other vein) from the vas deferens and/or artery;

(d) loop the spike around the deferential vein;

(e) coagulate the deferential vein;

(f) optionally open the spike loop; and

(g) remove spike from abdomen, optionally cutting the vein in the process.

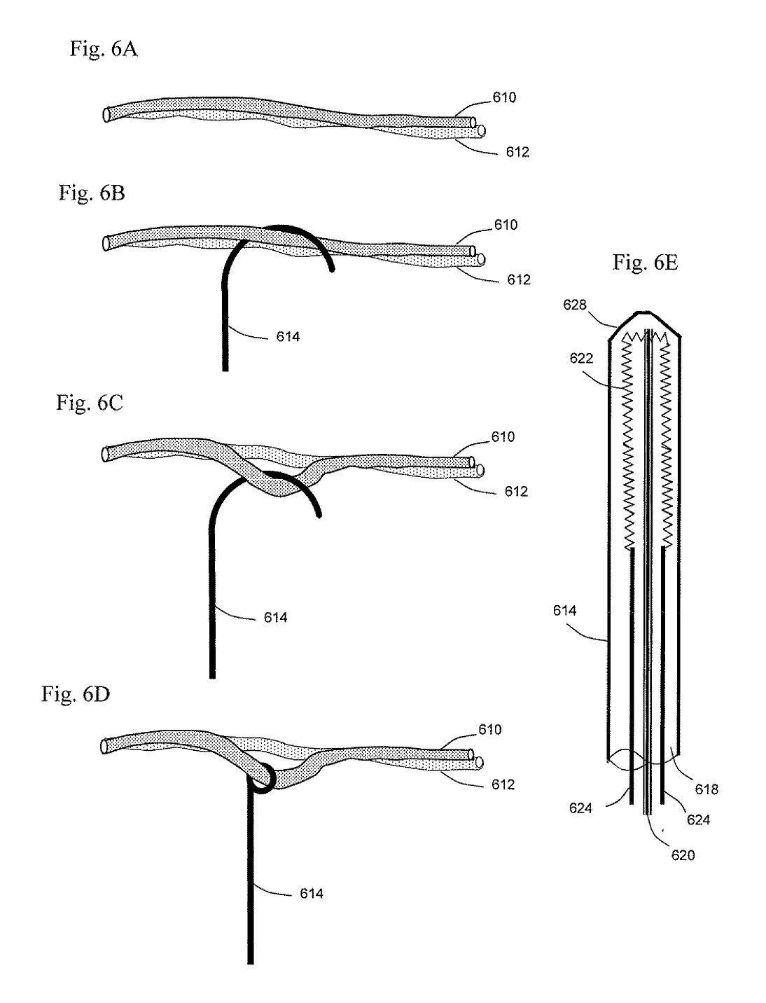

FIG. 6A schematically illustrates a section of deferential 610 vein (110) and a section of vas deferens 612 as they are attached to each other.

FIG. 6B schematically illustrates a spike 614 inserted between the deferential vein and the vas deferens, in accordance with some embodiments of the invention.

In some embodiments of the invention, spike 614 is introduced through the skin of the frontal pelvis into the abdomen in a procedure similar to laparoscopy, and inserted between deferential vein 610 and vas deferens 612. Optionally, spike 614 is curved at the distal end (towards the abdomen) so that it bends about the deferential vein 610. Optionally, spike 614 is flexible, at least at the distal end, to facilitate maneuvering in the abdomen and to bend about the deferential vein 610.

In some embodiments of the invention, a tube (e.g. a catheter) adapted to penetrate the abdomen is used as an auxiliary tool for the spike such that the tube is maneuvered to a location proximal to the deferential vein, and spike 614 is inserted in the lumen of the tube to reach the deferential vein. Optionally, the tube is removed before the separation or occlusion of the deferential vein, or optionally after the separation or occlusion of the deferential vein.

In some embodiments of the invention, as spike 614 is curled around deferential vein 610, it is pulled in order to separate the deferential vein 610 from vas deferens 612.

FIG. 6C schematically illustrates spike 614 separating deferential vein 610 from vas deferens 614, in accordance with some embodiments of the invention.

In some embodiments of the invention, spike 614 is configured to curl, so that it forms a loop around deferential vein 610, without touching vas deferens 614.

FIG. 6D schematically illustrates spike 614 curling into a loop around deferential vein 610 separated from vas deferens 614, in accordance with some embodiments of the invention.

In some embodiments of the invention, the shape of the distal end of spike 614 is controlled by twisting the proximal end or otherwise manipulating the proximal end of spike 614.

In some embodiments of the invention, spike 614 comprises a tube 614, optionally flexible at least about the distal section that is adapted to curl and loop. The section length is about 3 mm to about 15 mm, according to the operation procedure and vein. The section width is about 1 mm. optionally the width is less than about 1 mm, such as about 0.5 mm. Optionally, the section width is larger than about 1 mm, such as about 1.2 or about 1.5 mm.

In some embodiments of the invention, tube 614 has a tapered tip at one end (as illustrated for example by 628 in FIG. 6E) which is adapted to separate at least a part of the deferential vein 610 from the vas deferens.

In some embodiments of the invention, spike 614 comprises or is coated with a material for smooth movement in the abdomen and/or for avoiding damage to organs, and particularly, without limiting, avoiding damage to the delicate deferential vein and vas deferens.

In some embodiments of the invention, spike 614 comprises:

(a) tube 614 having a lumen 618; and

(b) a control wire 620;

FIG. 6E schematically illustrates a spike 614 having lumen 618 with control wire 620, in accordance with some embodiments of the invention.

In some embodiments of the invention, spike 614 is configured to curl by manipulating control wire 620 in lumen 618 from the proximal end. Optionally, control wire 620 is attached alongside spike 614. Optionally, spike 614 with lumen 618 comprises a shape memory alloy so that by flushing lumen 618 with a fluid in a suitable temperature spike 614 curls. Optionally, by adjusting the temperature the curvature is controlled. Optionally, spike 614 comprises a bi-metal construction at the distal end so that by flushing lumen 618 with a fluid in controlled temperature the curvature is controlled.

In some embodiments of the invention, a control wire 620 is used for maneuvering in the abdomen, and another control wire 620 is used to separate and/or loop spike 614. Optionally or additionally, another control wire 620 is used for looping the spike 614. The control wires or other wires may be used for the reverse operation for opening the loop and removing spike 614.

FIG. 6E further illustrates schematically spike 614 having lumen 618 with a filament 624 at the distal end connected to conductors 620, in accordance with some embodiments of the invention. In some embodiments of the invention, the conductors exit at the proximal end and can be connected to a current source.

In some embodiments of the invention, when spike 614 is looped (curled) around deferential vein 610, current is supplied via conductors 620 to filament 622 which heats up and coagulates deferential vein 610. Optionally, the current is supplied in a short burst or pulse such that deferential vein 610 is coagulated but the vein environment is not heated, at least avoiding a detrimental effect on other organs, particularly vas deferens 612.

In some embodiments of the invention, filament 622 and conductors 624 are eliminated, and spike 614 is adapted for high temperature sufficient to occlude the deferential vein. Optionally, lumen 622 is configured to fill with high temperature fluid so that that deferential vein 610 is occluded by thermally induced thrombosis. Optionally, a separate tube is inserted in lumen 622 and filled with hot fluid to apply the thermal occlusion.

In some embodiments of the invention, the spike 614 is adapted for low temperature such as that of liquid air or similar temperature, and filament 622 and conductors 624 are eliminated. Optionally, lumen 622 is configured to fill with low temperature fluid so that that the deferential vein 610 is occluded by cryogenic (freezing) induced thrombosis. Optionally, a separate tube is inserted in lumen 622 and filled with cold fluid to apply the cryogenic (hypothermal) occlusion.

In some embodiments, the spike is pre-configured to curl and prevented form doing so by an inner stylet or an outer over tube. Retracting the outer over tube and/or stylet, allows spike 614 to curl. Optionally or alternatively, spike 614 is curled by inserting a culling stylet into a lumen thereof. Optionally, a top of spike 614 is more flexible than a body thereof, so that the curling stylet can only cause curling at the tip.

In some embodiments of the invention, the coagulation procedure as described above is performed with imaging control. In some embodiments of the invention, the visual control is by x-ray imaging. Optionally, spike 614 comprises radio-opaque elements so that it is distinguished under x-ray imaging. Optionally, the operation is guided by ultra sound imaging, for example by using a TRUS (Trans-rectal Ultrasound) probe. Optionally, a fiber-optic tube is inserted into the abdomen in a laparoscopic procedure near spike 614, illuminating spike 614 and nearby organs, including deferential vein 610 and vas deferens 612, and the operation is controlled from a monitor. Optionally, spike 614 is colored such that is clearly distinguished relative to the environment.

### Subcutaneous Gripping and Injection

In some embodiments of the invention, deferential vein 110 is occluded by injecting a sclerosant agent into the vein in a in a procedure similar to laparoscopy. In some embodiments of the invention, deferential vein 110 or a branch of vesicular plexus 128 is held by a gripper (e.g., a pair of arms shaped for gripping a vessel) mounted on a tube and an injector is inserted in the tube to pierce deferential vein 110 and inject a sclerosant.

In some embodiments of the invention, in order to occlude deferential vein 110, the vein is optionally separated from the vas deferens in order not to damage the latter. Similarly, each branch of the plurality of branches of vesicular plexus 128 is optionally separated from the surrounding tissues.

In some embodiments of the invention, an appropriate apparatus is adapted to perform a combination of separation and occlusion. Optionally, the vein is secured so that an injector can safely penetrate the delicate vein without damaging other tissues or organs. Optionally, the equipment is configured to separate the vein, grip the vein, and provide a path to the vein from outside the abdomen.

For clarity and brevity in the following discussion, without limiting generality, deferential vein 720 (110) is referred to as an example of a vein branching from another vein and the vesicular vein is referred to as an example of a vein into which a branching vein opens, whereas the vas deferens represents also other tissues around the vesicular plexus.

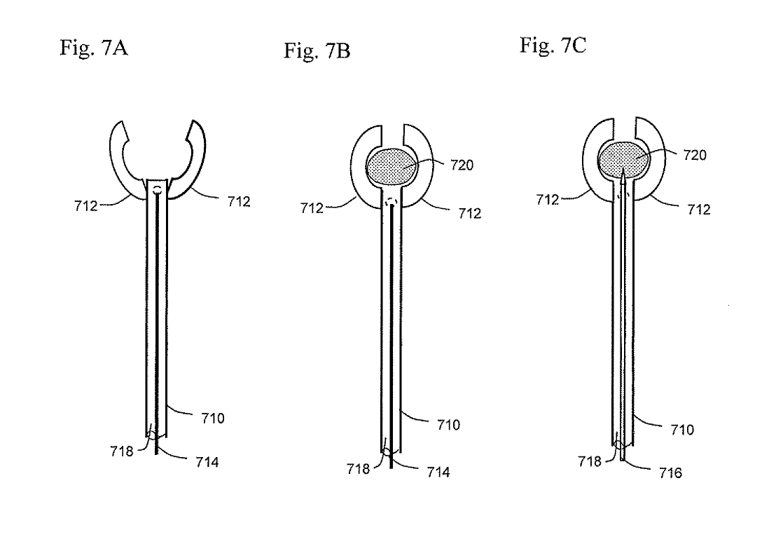

FIG. 7A schematically illustrates a tube 710 having a lumen 718 and a hinged pair of grippers 712 at the distal end (towards the vein), shown in open position, in accordance with some embodiments of the invention.

In some embodiments of the invention, grippers 712 have a rounded shape, optionally tapered at the distal end such as to enable separation of the deferential vein from other tissues. Optionally, the shape of grippers 712 on the side facing one another is rounded or shaped such as to grip a vein firmly but without destroying or damaging the vein in a manner which that would prevent the vein occlusion and/or cause hemorrhage.

In some embodiments of the invention, grippers 712 comprise or are coated with a slippery material for supporting movement in the abdomen and/or for avoiding damage to organs, and particularly, without limiting, avoiding damage to the delicate deferential vein and vas deferens. Optionally, the gripers define a space between them for holding a vein.

In some embodiments of the invention, the width of gripper 712 is adapted to insert between the deferential vein and connecting tissues such as the vas deferens. Accordingly, in some embodiments of the invention, the maximal width of gripper 712 is about 1 mm. Optionally, the maximal width of gripper 712 is larger then about 1 mm, such as about 1.5 mm. Optionally, the maximal width is smaller than about 1 mm such about as 0.8 mm.

In some embodiments of the invention, the length of gripper 712 is adapted to separate and grip the deferential vein. Accordingly, the length of gripper 712 is about 1 mm. Optionally, the length is larger than about 1 mm, such as about 1.5 mm or about 2 mm. Optionally, the length is smaller than about 1 mm, such as about 0.8 mm.

In some embodiments of the invention, the maximal width of the opening between grippers 712 in a closed configuration is adapted to grab a vein. Accordingly, in some embodiments of the invention, the maximal width of the opening is about 0.3 mm. Optionally, the maximal width less than about 0.3 mm such as about 0.2 mm. Optionally, the maximal width is larger than about 0.3 mm such as about 0.4 mm or about 0.5 mm.

The term ‘gripper’ denotes in the discussion, according to the context, a pair of grippers and optionally tube 710 with the mounted grippers 712.

In some embodiments of the invention, the procedure is controlled by the imaging methods described above.

To clarify the following procedure, it may be optionally summarized as follows:

(a) apply imaging (e.g. optical, ultra sound, x-ray);

(b) introduce a gripper mounted on a tube into the abdomen, under imaging control;

(c) insert a gripper jaw between a deferential vein and a vas deferens (and optionally artery);

(d) grip the deferential vein;

(e) insert an injector in the tube lumen;

(f) pierce the deferential vein;

(g) inject a sclerosant into the vein by the injector; and

(h) remove the injector and gripper.

In some embodiments of the invention, the gripper is inserted in a closed state, and opens to grab the vein and closes on it.

In some embodiments of the invention, gripper 712 is controlled by a control wire 714 inserted in lumen 718. Optionally, control wire 714 is installed in lumen 718 before the gripper is introduced into the abdomen. Optionally, control wire 714 is inserted in lumen 718 after the introduction of tube 710 into the abdomen and attaches to gripper 712 at near the distal end. Optionally or additionally, control wire is combined with tube 712 outside lumen 718, such as alongside tube 718.

In some embodiments of the invention, pulling control wire 714 from the proximal end closes the gripper. Optionally or alternatively, gripper 712 is closed by pushing control wire 714, or otherwise maneuvering the wire, such as by rotating the wire or with a handles that control the position of grippers 712 similar to scissors. Various gripper operating mechanisms are known in the art and may be used, for example, retracting the grippers relative to an over tube (not shown), with the grippers spring-loaded to open.

In some embodiments of the invention, tube 710 is introduced into the abdomen and inserted, typically with one gripper 712, between the deferential vein 720 and the vas deferens (see also FIG. 6A. to FIG. 6D) and grips vein 720.

In some embodiments of the invention, once gripper 712 is around the deferential vein 720, gripper 712 is closed around deferential vein 720.

FIG. 7B schematically illustrates tube 710 having lumen 718 and comprising hinged pair of grippers 712 shown in closed position around vein 720 at the distal end, in accordance with some embodiments of the invention.

FIG. 7C schematically illustrates tube 710 having lumen 718 and comprising hinged pair grippers 712 shown in a closed around vein 720 position at the distal end, and an injector 716 inserted in lumen 718 piercing vein 720, in accordance with some embodiments of the invention (guide wire 714 is not shown for clarity).

In some embodiments of the invention, injector 716 comprises a tube having a lumen wherein tube 716 comprises a pointed tip having an orifice. In some embodiments of the invention, the injector comprises a stopper, such as a ring near the tip, configured to prevent the injector from piercing through the vein. Optionally, the stopper is about 0.1 mm from the tip. Optionally, the stopper is about 0.2 mm from the tip. Optionally, the stopper is about 0.3 mm from the tip.

In some embodiments of the invention, when gripper 712 holds vein 720, gripper 712 is optionally maneuvered to a position appropriate for injection.

In some embodiments of the invention, injector 716 is inserted into lumen 718 and pushed against gripped vein 720, piercing into the vein. In some embodiments of the invention, when the injector tip is inside vein 720, a sclerosant agent is injected into vein 720 via injector 716.

In some embodiments of the invention, once the sclerosis operation is over, so injector 716 is pulled out of lumen 718. Subsequently, gripper 712 is opened, optionally by pushing or otherwise maneuvering control wire 714, such as by rotating the wire, releasing vein 720. Tube 710 and gripper 712 are pulled out of the abdomen, optionally in closed state. Optionally, injector 716 is pulled out with tube 710.

### Treatment of the Vas Deferens and Deferential Vein

In some embodiments of the invention, in some cases when separation of the deferential vein from the vas deferens is difficult and/or impractical, both vessels may be closed and/or occluded, particularly in life threatening condition such as a developed prostate cancer and/or metastases. In some cases, possibly in a non life threatening condition, when the subject is in an age where fertility is not important closing both the deferential vein and the vas deferens may be performed, particularly if the separation between them is difficult.

In some embodiments of the invention, closing the deferential vein and the vas deferens is carried out, at least partly, using procedures as described above.

### Subcutaneous Approach

In some embodiments of the invention, the subcutaneous approach (similar to laparoscopy) is performed via the abdomen wall, optionally near the groin or loins. Optionally, the approach is via a testis sack in an upward direction.

### Exemplary Catheterization and Occlusion of the Internal Spermatic Veins

The procedure is performed using a coaxial system, with a femoral vein sheath, two shaped guiding catheters, one for the left side and a different curve for the right, both designed to match the expected anatomy. Optionally, the procedure is performed with a 6 French sheath and 6 French guiding catheters through which the 3 French treatment catheter is placed.

Optionally or alternatively, the entire procedure is performed using a 3F infusion catheter with tandem balloons (a proximal and distal balloon) or a single proximal balloon 20-25 cm from its tip, and relying on the patient's compressing the vein in the groin. The 3 French catheter, maneuvered over a stiff 0.014-0.018″ wire with a variably curved, soft, flexible tip that could be used to directly engage the orifice of the ISV.

### Femoral Vein Approach

In a conventional method, namely, a femoral vein approach, a procedure in so accordance with an exemplary embodiment of the invention is performed as follows:

The localization of the right femoral vein puncture site is confirmed by physical examination—e.g., at or below a line from the anterior superior iliac spine to the symphysis pubis. After skin anesthesia with 1-2 ml local anesthetic, and optionally during a Valsalva maneuver to distend the femoral vein, the vein is punctured with an 18 g femoral puncture needle. Optionally, the needle is sheathed.

A venous puncture is first performed as a two wall puncture to allow an additional 1 ml local anesthesia to be placed deep into the vein. Upon entering the vein, the guidewire is brought through the needle into the vein and advanced into the pelvis. The needle or sheath is withdrawn around the guidewire while compressing the puncture site. The venous sheath is placed over the guidewire with its external port at the groin. The side port of the sheath is optionally flushed with heparinized saline solution, and this is optionally repeated throughout the procedure at 5-10 minutes intervals. The guiding catheter is advanced into the inferior vena cava (IVC) over the guidewire, and, under fluoroscopic control, maneuvered into the orifice of the left renal vein.

In about 5% of cases, the junction of the left renal vein with the IVC is an obtuse angle, heading caudally or cephalad from the kidney, and then the internal spermatic vein (ISV) orifice joins the left renal vein at an acute angle, making it difficult to enter from the upwards slanting renal vein.

In another 2-3% of cases, the ISV joins the renal vein together with or just below a paraaortic vein, and there may be difficulty in recognizing this situation, and identifying the orifice of the ISV. In such cases, the guiding catheter is maneuvered so that its tip overlies the orifice and, optionally, with gentle but firm rotation of the catheter, the orifice of the ISV is engaged. Optionally, the latter operation is aided by a combination of suspended respiration and tilting of the fluoroscopic table.

In some cases it may be necessary to engage the orifice just with the treatment catheter soft tip or with its flexible guidewire. Occasionally, a guiding catheter with an alternative tip (such as is used with adrenal vein or spinal artery) may be needed to engage the orifice of the ISV.

When the orifice of the left ISV is first encountered, it may have an orifice valve which is open or closed. The valve can usually be entered by tilting the patient head down and attempting to advance the inner (coaxial) 3F (1 mm) inner infusion catheter.

After entering the orifice of the ISV with the 3F infusion catheter, there may be additional valves all along the ISV which may be difficult to pass. The valves are optionally passed by a combination of patient/table positioning, breathing maneuvers, and applied suction via the guiding catheter. A balloon tipped catheter placed at the orifice to which suction is applied may be helpful in opening the valve and allowing the treatment catheter to cross it, by distending the vein. If the valves can be easily passed, then a 3F infusion catheter, optionally with occlusion balloon(s), is introduced with the 0.014 in guidewire in place. The treatment catheter is optionally maneuvered to the lowest desired point for sclerotherapy—optionally just above the inguinal ligament.

In order to visualize the ISV anatomy, intravenous contrast is injected into the catheter through the central port hub under fluoroscopic control and images are obtained of the ISV, both distally and proximally, using appropriate manipulation of the tilting fluoroscopy table to establish the anatomy of the ISV and its communicating and collateral tributaries. Radiology imaging is used to identify both collateral ISV tributaries as well as intercommunications between the ISV and other retroperitoneal veins, and whether there are evident communications to other significant veins, such as the renal capsular vein, the ureteral vein and paravertebral retroperitoneal veins.

Optionally, once the anatomy of the ISV has been established, the therapeutic phase is begun.

The sclerotherapy treatment optionally covers the length of the ISV from just above the inguinal ligament until within 3-5 cm of the ISV orifice. Optionally, the treatment comprises segmental injection of sclerosant while the ISV is compressed in the inguinal region to prevent reflux of sclerosant downwards and flushing out of the sclerosant upwards with the venous blood flow. With a treatment catheter fitted with one or two occlusion balloons, the efficacy of isolation of the segment of the ISV is improved and the sclerosing process can be performed in fewer steps, saving sclerosant and time.

For the sclerosis operation, a mixture of up to 2 ml sclerosing agent with 0.25-0.5 ml 2% local anesthesia, such as lidocaine, is optionally made and agitated in a 3-5 ml plastic syringe. It may be transferred from syringe to syringe using a stopcock arrangement to produce foam. The amount of sclerosant agent depends on the size of the ISV.

If the treatment catheter has a distal balloon, it is optionally gently inflated to occlude the vein. Other occluding means may be used as well. Occlusion is optionally confirmed by a small amount of intravenous contrast being injected through the side hole of the treatment catheter. Optionally, when there is a proximal balloon on the treatment catheter (or on the end of the guiding catheter) it is gently inflated to occlude the upper portion of the ISV. Repeated control injection of intravenous contrast is optionally provided until there is no reflux of contrast material into the veins below the inguinal region (e.g., towards the scrotum) indicating thorough occlusion of the ISV continuation into the scrotum.

In an exemplary embodiment of the invention, a small syringe with 0.54.0 ml intravenous contrast material is prepared. Half is injected into the treatment catheter. It is observed under fluoroscopy to make sure that it does not continue below the inguinal region towards the scrotum. If it does, then the occlusion balloon (if present) is repositioned/re-inflated or digital compression of the groin is adjusted until there is no contrast which passes the inguinal region. Then, about 1-2 ml of the sclerosant mixture is injected. The tip of the treatment catheter is withdrawn 10-15 cm, and the injected material is allowed to remain in the vein.

The fluoroscopy table can be tilted slightly, “feet down”, up to 10 degrees, but usually after the injection and the 5-10 minutes afterwards the table is kept in horizontal position. Optionally, tilting is used to control a flow direction of sclerosing or other occlusion enhancing or causing material.

In an exemplary embodiment of the invention, five to ten minutes after the injection, the contents of the treated vein are aspirated through the catheter and discarded. The catheter is flushed with a small amount of saline or intravenous contrast. Then, the ISV is optionally examined using intravenous contrast material with the patient in the semi-erect position (20-50 degrees) to confirm vein occlusion and identify any collateral veins which appear following occlusion of the main ISV.

If needed, a second segment of vein is treated in the same manner, until only a 5 cm “stump” of the ISV remains visible as including contrast material inflow.

If the ISV is adequately occluded, the guiding catheter is optionally flushed with sterile saline solution, and a semi-erect contrast injection is made into the left renal vein to help search for any collateral veins that fill the ISV that may not have been visible before the main ISV had been occluded. When it is determined that there is no filling of the left ISV directly or indirectly, then the same guiding catheter used for the left side is maneuvered to the right renal vein (Rt RV) and a diagnostic venogram is performed in the semi-erect position with a hand injection, looking for the number of right renal veins and their connections to the IVC, as well as for a variant Rt ISV configuration which joins one of the right renal veins directly instead of the ISV (usually the ISV will enter the IVC at the level of L2, though with 10-20 percent, there may be multiple connections between the Rt ISV and the IVC.

At this stage, the left sided guiding catheter is optionally exchanged for the right sided guiding catheter whose tip is re-configured inside the IVC and brought to lie above the expected orifice of the Rt ISV.

On the right, there may be a problem identifying the junction of the ISV with the IVC. There may be a valve at the orifice of the Rt ISV which makes entering it difficult.

In these cases, the use of table positioning (“feet elevated” [Trendelenberg]), and breathing maneuvers, may help.

Once the Rt ISV is entered the inner catheter needs to be advanced into the right ISV. Similar to the left side operation, there may be valves that need to be crossed, using the same maneuvers and/or devices as described on the left. The remainder of the treatment is performed in the same fashion as on the left.

At the conclusion of the treatment, when the Rt ISV appears occluded and no collateral filling (“bypass”) of the Rt ISV is seen, the tandem catheter is withdrawn, the guiding catheter is pushed upwards to dislodge it from the Rt ISV orifice, turned and straightened by either maneuvering the tip into the left renal vein or downwards into the orifice of the left internal iliac vein, the guiding catheter is removed, the femoral vein sheath is removed and the puncture site is compressed for 5 minutes before allowing the patient to careful sit up while compressing the groin and begin his recovery.

### Arm Vein Approach Exemplary Embodiment

In an exemplary embodiment of the invention, a direct venous puncture with an approach identical to PICC line insertion is used. A 100-120 cm long 4 French catheter shaped like a “Head Hunter” catheter with a hydrophilic tip is inserted either directly into the vein or through a 4F sheath. Optionally, a single type of catheter is used to enter right and left sides. A flexible guidewire, possibly with hydrophilic coating and/or an “extra-soft” tip (like a “Bentson” wire) is used to advance the 4F catheter into the right ISV for sclerotherapy. Optionally, a coaxial system is used and the guiding catheter comprises an inflatable balloon to permit occlusion of the upper orifice while the sclerosant is injected through the inner sclerotherapy catheter. Optionally or alternatively, the method does not use the coaxial system, and the treatment catheter comprises an occlusion balloon about 20-25 cm above the catheter tip to occlude the lumen of the ISV, and a second occlusion balloon in the distal portion of the sclerotherapy catheter. The sclerosant is injected via a side hole in the portion of the treatment catheter between the two balloons (in case of two balloons), or 3-5 cm from its distal end. Once positioned in the ISV, as in the femoral vein approach, the sclerotherapy is performed in the same manner.

After the sclerotherapy treatment of the right side, the left side is treated using the same shaped guiding catheter to access the left ISV and catheterize it as described above for the femoral vein approach.

### Internal Jugular Approach

In an exemplary embodiment of the invention, a puncture performed in the right internal jugular in the usual fashion with local anesthesia with or without ultrasound guidance. A 4 or 5 French sheath is placed in the internal jugular vein, and the continuation of the procedure is performed in the same manner as the arm vein approach described above.

### Reaching the Deferential Vein and the Vesicular Plexus

The deferential vein 110 connects at one side to pampiniform plexus 118 and at the other side to vesicular vein 112. Vesicular plexus 128 connects prostate 120 to vesicular vein 114. In some cases vesicular plexus 128 has a plurality of branches connecting to vesicular vein 112.

In exemplary embodiments of the invention, an approach is provided to reach the junction of deferential vein 110 and pampiniform plexus 118, for example, one or more of:

### Femoral Vein Approach

Femoral vein to external iliac vein, to common iliac vein, to the internal iliac vein, to the vesicular vein, reaching the opening of the deferential vein and the opening of the vesicular plexus.

### Arm Vein Approach

Arm vein to superior vena cava, to inferior vena cava, to common iliac vein, to the internal iliac vein, to the vesicular vein, reaching the opening of the deferential vein and the opening of the vesicular plexus.

### Jugular Vein Approach

Jugular vein to superior vena cava, to inferior vena cava, to common iliac vein, to the internal iliac vein, to the vesicular vein, reaching the opening of the deferential vein and the opening of the vesicular plexus.

### Left Internal Spermatic Vein Approach

Vena cava to left renal vein, to left internal spermatic vein, to the pampiniform plexus, reaching the opening of the deferential vein. The inferior vena cava is accessed via the femoral vein or arm vein or jugular vein as described above.

### Right Internal Spermatic Vein Approach

Inferior vena cava to right internal spermatic vein, to the pampiniform plexus, reaching the opening of the deferential vein. The inferior vena cava is accessed via the femoral vein or arm vein or jugular vein as described above.

### EXAMPLES

The following examples should be considered a presentation of results that do not necessarily limit the scope of the invention. Two studies were conducted.
Study 1. 45 patients who suffer from PCa for were randomly selected and checked for varicocele. The diagnosis was made by physical examination and contact thermography, using a flexible liquid crystal thermostrip (FertiPro® by Breemen, Belgium), which is considered most accurate and sensitive for detection of bilateral varicocele.
Study 2. 7 men, 63-76 years of age, who suffered from localized PCa were treated. Treatments were performed by venography and percutaneous selective sclerotherapy of the ISVs and all associated bypasses and retroperitoneal collaterals. The procedure used is described in Gat Y., Chakraborty, J., Zukerman, Z., Gornish, M. Varicocele, Hypoxia, and Male Infertility. Fluid mechanics analysis of the impaired testicular venous drainage system. Hum. Reprod. (2005); 20:2614-2619. Editorial Comment in J. Urol. (2006), Apr. 17(4), 1454.

This technique eliminates the pathologic hydrostatic pressure in the impaired testicular drainage system. The study was carried out in accordance with the principles of the Declaration of Helsinki, approved by the ethical committee of the hospital. All patients gave a written informed consent to participate in the study prior to the procedure. The treatments were performed by a highly experienced interventional radiologist (a specialist in venography of the male pelvis) in an interventional radiology suite equipped with digital fluoroscopic imaging (and a 45/90 degree tilt table).

### Results

Re study 1. All the patients that suffered from PCa in various degrees were found to have bilateral varicocele.

Re study 2. Before treatment all patients reported nocturia, with an average of 4 times per night (ranging from three to seven). Prostate volume was determined by transabdominal ultrasonography, with an average of 61.4 ml (range 41-114 ml), and PSA average level was 9.16 ng/ml (range 3.7-13 ng/ml). Following the treatment (4-12 weeks later), the prostate volume decreased to an average of 37 ml (range 24-71 ml), nocturia decreased to an average of one per night (range 0-2 per night), and PSA average level decreased to 5.7 ng/ml (range 2.24-9.5 ng/ml). The results are summarized in Table 1. Following 6 months after the treatment no prostate cancer cells were found by repeated biopsies in 2 patients that suffered before the treatment from localized adenocarcinoma of prostate, Gleason 6 (3+3). One patient retained cancerous cells.

TABLE 1
Prostate volume, PSA levels and Nocturia before and after treatment in 7
patients with localized Prostate cancer and bilateral varicocele. Ages were
between 60 and 79 for all patients. Volume is in ml, PSA in ng/ml. Nocturia as
a count

| | Volume | volume | PSA | PSA | Nocturia | Nocturia | | Bx. |
|---|---|---|---|---|---|---|---|---|
| Patients | before | after | before | after | before | after | Bx. Before | After |
| 1 | 41.7 | 24 | 14.5 | 5 | 3 | 1 | Adeno Ca | None |
| | | | | | | | Gleason 6 | |
| | | | | | | | (3 + 3) | |
| 2 | 114.5 | 71.4 | 10.5 | 9.5 | 3 | 1 | Adeno Ca | Ad Ca 6 |
| | | | | | | | 6 (3 + 3) | (3 + 3) |
| 3 | 62.98 | 26.23 | 9 | 3 | 7 | 2 | Adeno Ca | |
| | | | | | | | 6 (3 + 3) | |
| 4 | 61.53 | 34 | 8.45 | 6.99 | 3 | 1 | Adeno Ca | |
| | | | | | | | 6 (3 + 3) | |
| 5 | 50.88 | 33.08 | 8.8 | 7.8 | 4 | 1 | Adenod Ca 6 | None |
| | | | | | | | (3 + 3) | |
| 6 | 43 | 27.39 | 3.69 | 2.24 | 3 | 1 | Adeno Ca | |
| | | | | | | | 6 (3 + 3)1/9 | None |
| 7 | 79 | 54.14 | 6.9 | 7.2 | 5 | 2 | Adeno Ca | |
| | | | | | | | 6 (3 + 3) | |
| Average | 64.80 | 38.61 | 8.83 | 5.96 | 4.00 | 1.29 | | |

### General

Various of the apparatus described herein may be provided in kit form, optionally with instructions and/or materials for treatment and/or vessel plugs.

All trademarks are the property of their respective owners.

In the description and claims of the present application, each of the verbs “comprise”, “include” and “have” as well as any conjugates thereof, are used to indicate that the object or objects of the verb are not necessarily a complete listing of members, components, elements or parts of the subject or subjects of the verb.

As used herein, the singular form “a”, “an” and “the” include plural references unless the context clearly dictates otherwise. For example, the term “a procedure” or “at least one procedure” may include a plurality of compounds, including mixtures thereof.

As used herein, the term “treating” includes abrogating, substantially inhibiting, slowing and/or reversing the progression of a condition, substantially ameliorating clinical and/or aesthetical symptoms of a condition and/or substantially preventing and/or delaying the appearance of clinical and/or aesthetical symptoms of a condition.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals in between.

As used herein the term “about” refers to ±10% of the referenced numerical value, unless otherwise specified.

The present invention has been described using detailed descriptions of embodiments thereof that are provided by way of example and are not intended to necessarily limit the scope of the invention. In particular, numerical values may be higher or lower than ranges of numbers set forth above and still be within the scope of the invention. The described embodiments comprise different features, not all of which are required in all embodiments of the invention. Some embodiments of the invention utilize only some of the features or possible combinations of the features. Alternatively and additionally, portions of the invention described/depicted as a single unit may reside in two or more separate physical entities which act in concert to perform the described/depicted function. Alternatively and additionally, portions of the invention described/depicted as two or more separate physical entities may be integrated into a single physical entity to perform the described/depicted function. Variations of embodiments of the present invention that are described and embodiments of the present invention comprising different combinations of features noted in the described embodiments can be combined in all possible combinations including, but not limited to use of features described in the context of one embodiment in the context of any other embodiment. The scope of the invention is limited only by the following claims.

All publications and/or patents and/or product descriptions cited in this document are fully incorporated herein by reference to the same extent as if each had been individually incorporated herein by reference or if they were reproduced in full herein.

1. A method for treating the prostate, comprising:
(a) positing the existence detecting abnormal cells that respond to an androgen in the prostate; and
(b) occluding an internal spermatic vein.

2. A method according to claim 1, wherein the abnormal cells are localized in the prostate.

3. A method according to claim 1, wherein the androgen comprises testosterone.

4-5. (canceled)

6. A method according to claim 1, further comprising administration of antiandrogen medication.

7. A method according to claim 6, wherein the antiandrogen medication is administered locally to the prostate.

8. A method according to claim 6, wherein the administration of antiandrogen follows the occlusion.

9. A method according to claim 1, wherein occlusion of an internal spermatic vein comprises occlusion a junction between the internal spermatic vein and a pampiniform plexus.

10. A method according to claim 1, further comprising application of at least one of chemotherapy, radiotherapy, brachytherapy, thermotherapy or cryotherapy.

11. A method for treating metastases, comprising:
(a) detecting prostate cancer metastases; and
(b) reducing venous pressure in the prostate while blocking reflux from testicular veins subject to abnormally high pressure.

12. A method according to claim 11, wherein reducing pressure in the prostate comprises occluding a vein between the prostate and a pampiniform plexus.

13. A method according to claim 12, wherein the vein is a least one of a deferential vein or a vesicular plexus vein.

14. A method according to claim 11, wherein an abnormally high pressure is maintained in the internal spermatic veins.

15. A method according to claim 11, further comprising administration of antiandrogen medication.

16. A method according to claim 15, wherein the administration of antiandrogen follows the blocking.

17. A method according to claim 11, further comprising application of at least one of chemotherapy, radiotherapy, brachytherapy, thermotherapy or cryotherapy.

18. A method for a preventive treatment of the prostate, comprising:
(a) assessing a risk for development of prostate disorder; and
(b) responsive to the risk, preventing venous reflux to the prostate.

19. A method according to claim 18, wherein preventing venous reflux to the prostate comprises occluding an internal spermatic vein.

20. (canceled)

21. A method according to claim 18, wherein prostate disorder comprises one of BPH and cancer.

22. A method according to claim 18, wherein the assessing comprises assessing varicocele.

23. A method according to claim 22, wherein varicocele is assessed by testicular temperature.

24. A method according to claim 18, wherein assessing comprises determining at least one of PSA level, prostate size or venous anatomy.

25. An intravascular catheter configured to apply material sideways from a first vein into a second vein branching from the first vein, comprising:
(a) a tube having a lumen; and
(b) an orifice connecting the lumen and a longitudinal external side of the tube.

26. A catheter according to claim 25, wherein the connection is substantially perpendicular to the longitudinal external side of the tube.

27. A catheter according to claim 25, wherein the lumen is sealed at the distal end.

28. A catheter according to claim 27, wherein the orifice is sufficiently close to the sealed end to enable material in the lumen to deflect from the lumen via the orifice outside the lumen.

29. A catheter according to claim 25, further comprising at least one guiding element configured to support a positioning of the orifice in front of an opening of the second vein.

30. A catheter according to claim 29, wherein the at least one guiding element comprises a guide wire in the lumen movable into the orifice and the opening of the second vein.

31. A catheter according to claim 29, wherein the at least one guiding element comprises at least one radio opaque element located about the perimeter of the orifice.

32. A catheter according to claim 25, further comprising at least one expandable and retractable element capable of blocking material drainage along the outside of the catheter.

33. A catheter according to claim 32, wherein the expanded at least one element is configured to fixate the catheter to walls of the first vein.

34. A catheter according to claim 25, wherein the material comprises a sclerosant.

35. A catheter according to claim 25, wherein the material comprises a coil.

36-61. (canceled)

62. Use of an anti-androgen composition for treatment of prostate cancer in patients with a blocked differential vein.

63. Use of a sclerosant for treatment of prostate cancer or BPH.

64. Use of an anti-androgen composition for treatment of prostate cancer by achieving a prostatic androgen level of less than 20% of a normal level.

65-67. (canceled)
